(19) United States
(12) Patent Application Publication
(10) Pub. No.: US 2020/0179294 A1
(43) Pub. Date: Jun. 11, 2020

(54) MANGANOPORPHYRIN-POLYPHENOL MULTILAYER CAPSULES AS RADICAL AND REACTIVE OXYGEN SPECIES SCAVENGERS

(71) Applicant: THE UAB RESEARCH FOUNDATION, Birmingham, AL (US)

(72) Inventors: Eugenia Kharlampieva, Birmingham, AL (US); Veronika Kozlovskaya, Birmingham, AL (US); Hubert Tse, Hoover, AL (US)

(21) Appl. No.: 16/210,390

(22) Filed: Dec. 5, 2018

(51) Int. Cl. A61K 9/48; A61K 35/39; A61K 47/54; A61K 47/69

(52) U.S. Cl. CPC A61K 9/4891; A61K 9/4816; A61K 9/4808; A61K 47/6943; A61K 35/39; A61K 47/546; A61K 9/4833

(57) ABSTRACT

The present disclosure provides layer-by-layer compositions comprising a plurality of polymer bilayers. Each bilayer comprises a poly(N-vinylpyrrolidone) hydrogen-bonded a polyphenol (tannic acid) layer. In the compositions, at least one poly(N-vinylpyrrolidone) has conjugated thereon a plurality of manganoporphyrin moieties. The layer-by-layer compositions can be deposited on the cell aggregate surface or deposited on an underlying surface such as that of a silica core which, when removed creates a capsule having a hollow space for the addition of such as a therapeutic agent.

### STATEMENT ON FUNDING PROVIDED BY THE U.S. GOVERNMENT

This invention was made with government support under NSF Grant DMR 1608728 awarded by the U.S. National Science Institute and NIH Grant Nos.: DK099550 awarded by the U.S. National Institutes of Health of the United States government. The government has certain rights in the invention.

### FIELD OF THE DISCLOSURE

The present disclosure is generally related to coatings applied to cells or aggregates of cells by hydrogen bonding to the cell surface. The present disclosure further relates to coated cells or aggregates of cells wherein the viability and functionality of the cells are retained.

### BACKGROUND

Reactive oxygen species (ROS) are important for cellular signaling and defense against microbial pathogens, but can directly cause damage to DNA, lipids, and proteins through free-radical mediated oxidative stress. Oxidative stress can facilitate tissue damage and cell death in a variety of human diseases including diabetes, atherosclerosis, Alzheimer's disease, kidney disease and cancer (Roberts et al., (2009) Life Sci. 84: 705-712; Thayer et al., (2011) Diabetes 60: 2144-2150; Delmastro-Greenwood et al., (2014) Antioxidants & Redox Signaling 20: 2465-2477). Imbalanced ROS in the body are managed by endogenous enzymatic antioxidants, including superoxide dismutases (SOD), which can dismutate the superoxide radical (O₂⁻) into hydrogen peroxide (H₂O₂), and catalase (CAT), which dissipates hydrogen peroxide into water and oxygen (Nimse & Pal (2015) RSC Adv. 5: 27986-28006; Limon-Pacheco & Gonsebatt (2009) Mutant. Res. 674: 137-147).

Exogenous metalloporphyrins can mimic the catalytic redox activity of CAT and SOD and diminish oxidative stress by antioxidant and immunomodulatory actions (Day et al., (1997) Arch. Biochem. Biophys. 347: 256-262; Asayama et al., (2007) Mol. Pharmaceutics 4: 484-486; Tse et al., (2004) Free Radic. Biol. Med. 36: 233-247; Jaramillo et al., (2009) Cancer Res. 69: 5450-5457; Ye et al., (2009) Free Radic. Biol. Med. 47: 786-793; Spasojevic et al., (2006) J. Inorg. Biochem. 100: 1897-1902). The most effective Mn (III) porphyrins can scavenge a broad range of oxidants such as superoxide, hydrogen peroxide, peroxynitrite, and lipid peroxyl radicals (Ferrer-Sueta et al., (2003) J. Biol. Chem. 278: 27432-27438; Day et al., (1999) Free Radic. Biol. Med. 26: 730-736). The utility of catalytic metalloporphyrin antioxidants to ameliorate inflammatory-mediated processes has been demonstrated in an adoptive transfer model of Type 1 diabetes (T1D) (Piganelli et al., (2002) Diabetes 51: 347-355), endotoxic shock (Zingarelli et al., (1997) Br. J. Pharmacol. 120: 259-267), protection of neuronal cells from apoptosis (Patel, M. (1998) J. Neurochem. 71: 1068-1074), inhibition of lipid peroxidation (Day et al., (1999) Free Radic. Biol. Med. 26: 730-736) and blocking of hydrogen peroxide-induced mitochondrial DNA damage (Milano & Day (2000) Nucleic Acids Res. 28: 968-97).

Local modulation of the oxidative stress is advantageous over global ROS suppression as ROS are crucial in many biochemically important events including cell-to-cell communication, cellular differentiation, apoptosis, and defense against pathogens. Local suppression of pro-inflammatory ROS has been shown through conjugation of SOD to nanocarriers (Hu & Tirelli (2012) Bioconjugate Chem. 23: 438-449), or embedding in/copolymerization of SOD mimetics into polymer matrices (Hume & Anseth (2011) J. Biomed. Mater. Res., 99A, 29-37; Arai et al., (2012) ACS Appl. Mater. Interfaces 4: 5453-5457). However, protein molecules may suffer from limited in vivo stability and be potentially immunogenic, while polymerizable SOD mimetics may involve harsh reactants and reaction conditions (Cheung et al., (2008) Adv Func. Mater. 18: 3119-3126). Conversely, non-complexed natural or synthetic small molecule antioxidants may suffer from low bioavailability (Saba et al., (2007) Free Radic. Biol. Med. 42: 1571-1578), elution from the parent matrices (Zhou et al., (2013) ACS Appl. Mater. Interfaces, 5: 3541-3548), and various degrees of cytotoxicity when diffusing into neighboring biological locales.

Nano-engineered coatings with non-covalent inclusion of ROS scavengers have demonstrated success for localized ROS scavenging. For instance, 5-μm microcapsules produced via multilayer assembly of ionically paired poly(styrene sulfonate) and poly(allylamine hydrochloride) (PSS/PAH) with embedded 4-nm iron oxide nanoparticles or CAT were moderately effective in reducing oxidation of encapsulated bovine serum albumin by hydrogen peroxide (Shchukin et al., (2004) Chem. Mater. 16: 3446-3451). Similarly, 21-nm polyelectrolyte complexes of CAT and cationic block polyethyleneimine-poly(ethylene glycol) increased the catalytic degradation of H₂O₂ (Zhao et al., (2011) Nanomedicine (Lond) 6: 25-42).

Among natural antioxidants, tannic acid (TA) has been widely exploited for the design of nanoengineered coatings and biomedical applications due to its ability to participate in ionic pairing (Shutava et al., (2005) Macromolecules 38: 2850-2858), hydrogen bonding (Erel-Unal & Sukhishvili (2008) Macromolecules 41: 3962-3970; Kozlovskaya et al., (2010) Soft Matter 6: 3596-3608), and metal coordination (Ejima et al., (2013) Science, 341: 154-157; Rahim et al., (2016) Angew. Chem. Int. Ed. 55: 13803-13807) because of its 25 phenolic groups on digalloyl ester branches connected to a glucose core (Kozlovskaya et al., (2012) Adv. Funct. Mater. 22: 3389-3398; Chen et al., (2013) Biomacromolecules 14: 3830-3841; Dierendonck et al., (2014) Adv. Funct. Mater. 24: 4634-4644; Zhuk et al., (2014) ACS Nano 8: 7733-7745; Driver et al., (2013) Eur. Polym. J., 49: 4249-4256; Shukla et al., (2012) Adv. Mater. 24: 492-496; Chen et al., (2017) ACS Nano 11: 3135-3146; Kozlovskaya et al., (2017) Biomacromolecules DOI: 10.1021/acs.biomac.7b00687).

Hydrogen-bonded multilayers of TA and non-ionic poly(N-vinylpyrrolidone) (PVPON) are non-toxic for conformal coating of pancreatic islets and helped them maintain in vitro and in vivo function (Rahim et al., (2016) Angew. Chem. Int. Ed. 55: 13803-13807; Pham-Hua et al., (2017) Biomaterials 128: 19-32). By disproportionating ROS, a (PVPON/TA) multilayer can affect the activation of redox-dependent signaling pathways that contribute to the synthesis of pro-inflammatory cytokines and chemokines that result in attenuated adaptive immune T cell effector responses involved in autoimmune activity and islet graft rejection (Rahim et al., (2016) Angew. Chem. Int. Ed. 55: 13803-13807; Kozlovskaya et al (2015) Adv. Healthcare Mater. 4: 686-694). Disproportionation of free radicals with (PVPON/TA) coatings also suppressed the activation of pro-inflammatory macrophages which are key for destruction of insulin-producing pancreatic β-cells in Type 1 Diabetes (T1D) (Pham-Hua et al., (2017) Biomaterials 128: 19-32).

Despite the antioxidant potential of the (PVPON/TA) coating system, the TA can lose its ROS-scavenging capability due to formation of quinones upon oxidation by radicals. Because of the quinone transformation, the coating integrity might be lost with time due to competing interactions of the TA phenol groups with free radicals versus H-bonds with PVPON. To mediate these drawbacks, we have designed a synergistic polyphenol-manganoporphyrin polymeric coating using covalent coupling of the SOD-mimetic manganoporphyrin modality to a PVPON copolymer which can be assembled with TA into an efficient ROS-scavenging (TA-manganoporphyrin-PVPON) multilayer coating. In contrast to a traditional approach, where metalloporphyrins have been simply added to solution or non-covalently embedded into polymer matrices, covalent inclusion of the manganoporphyrin as a pendant group should allow for highly controllable ROS modulation.

The stability of manganoporphyrin-PVPON (MnP-PVPON) in aqueous solutions and the effect of pH conditions on its assembly with TA using in-situ ellipsometry and surface wettability measurements was explored. Possible cytotoxicity of manganoporphyrin-PVPON/TA multilayer capsules (due to cationic pyridinium groups and manganese cations in the porphyrin molecule) was tested by treatment of splenocytes from non-obese diabetic (NOD) mice with various capsule concentrations for 48 hours. The effect of the inclusion of the MnP-PVPON instead of PVPON, the effect of the location of MnP-PVPON layers within the capsule shell, and the overall number of functional MnP-copolymer layers present in the capsule shell on the radical scavenging properties of the manganoporphyrin-polyphenolic hollow capsules compared to polyphenolic (PVPON/TA) capsules was also studied. We analyze the shell thickness of pure polyphenolic (PVPON/TA) and the manganoporphyrin-polyphenolic capsules using small-angle neutron scattering (SANS) measurements of the capsules in solution before and after free radical treatment were compared.

### SUMMARY

One aspect of the present disclosure provides embodiments of a multilayered composition comprising a plurality of polymer bilayers, wherein the polymer bilayers each comprises a poly(N-vinylpyrrolidone) (PVPON) polymer layer and a polyphenolic tannin layer, wherein the poly(N-vinylpyrrolidone) (PVPON) polymer layer and the polyphenolic tannin layer are hydrogen-bonded, and wherein at least one poly(N-vinylpyrrolidone) (PVPON) layer comprises a plurality of manganoporphyrin moieties conjugated to the poly(N-vinylpyrrolidone) (PVPON) layer.

In some embodiments of this aspect of the disclosure, the multilayered composition is configured as a multi-layered capsule defining a volume or is disposed as a multilayered coating on a support surface.

In some embodiments of this aspect of the disclosure, the multilayered composition can comprise between about 3 polymer bilayers and about 10 polymer bilayers.

In some embodiments of this aspect of the disclosure, the polymer layer proximal to the defined volume or proximal to the support surface can be a poly(N-vinylpyrrolidone) (PVPON) layer.

In some embodiments of this aspect of the disclosure, at least one poly(N-vinylpyrrolidone) (PVPON) layer is embedded within the multilayered composition and comprises a plurality of manganoporphyrin moieties conjugated thereto.

In some embodiments of this aspect of the disclosure, the multilayered composition further comprises a poly(N-vinylpyrrolidone) (PVPON) layer disposed on the plurality of polymer bilayers as a layer most distal from the defined encapsulated volume or from the support surface and further comprising a plurality of manganoporphyrin moieties conjugated thereto.

In some embodiments of this aspect of the disclosure, the multilayered composition can further comprise an animal cell or aggregate of animal cells within the volume defined by the multi-layered capsule.

In some embodiments of this aspect of the disclosure, the polymer layer of the multi-layered capsule proximal to the defined volume of the capsule is hydrogen bonded to the cell membrane of a cell or a plurality of cells within the volume defined by the multi-layered capsule.

In some embodiments of this aspect of the disclosure, at least one polymer layer of the multilayered composition can further comprise a functional moiety attached thereto, and wherein the functional moiety can be selected from the group consisting of: a detectable moiety, an immunomodulatory molecule, a growth factor, or a combination thereof, and wherein the at least one polymer layer optionally comprises a molecular group suitable for attaching the functional moiety to the polymer layer.

In some embodiments of this aspect of the disclosure, the multi-layered capsule can further comprise a pharmacologically active compound that reacts with a reactive oxygen species when delivered to a recipient animal or human subject.

In some embodiments of this aspect of the disclosure, the compound that reacts with a reactive oxygen species can be a pharmacologically active agent.

In some embodiments of this aspect of the disclosure, the multilayered composition can be admixed with a pharmaceutically acceptable carrier.

Another aspect of the disclosure encompasses embodiments of a method of encapsulating an isolated cell or cell aggregate, the method comprising the steps of: (a) providing an isolated cell or cell aggregate; and (b) encapsulating the isolated cell or cell aggregate by depositing a plurality of polymer bilayers, wherein each polymer bilayer comprises a poly(N-vinylpyrrolidone) (PVPON) polymer layer and a polyphenolic tannin layer, wherein the poly(N-vinylpyrrolidone) (PVPON) polymer layer and the polyphenolic tannin layer are hydrogen-bonded, and wherein at least one poly(N-vinylpyrrolidone) (PVPON) layer comprises a plurality of manganoporphyrin moieties conjugated to the poly(N-vinylpyrrolidone) (PVPON) layer, wherein the deposited plurality of polymer bilayers can form a capsule encapsulating the isolated cell or cell aggregate.

In some embodiments of this aspect of the disclosure, the polymer layer in contact with the isolated cell or cell aggregate is poly(N-vinylpyrrolidone) hydrogen-bonded to the outer surface of the cell or aggregate of cells, or to an extracellular matrix component of the cell aggregate.

In some embodiments of this aspect of the disclosure, the method can further comprise the step of attaching at least one functional moiety to the capsule encapsulating the cells or cell aggregate.

In some embodiments of this aspect of the disclosure, the functional moiety can be selected from the group consisting of: a detectable moiety, an immunomodulatory molecule, a growth factor, a cell receptor ligand, a polypeptide cell receptor, or any combination thereof.

In some embodiments of this aspect of the disclosure, the cell or cell aggregate can be selected from the group consisting of: a bacterial cell, a viral cell, a plant cell, an artificial cell, and an animal cell, and wherein the cell or aggregate of cells is an isolated cell or aggregate of cells or a cultured cell or aggregate of cells.

In some embodiments of this aspect of the disclosure, the cell aggregate can be an isolated human or animal pancreatic islet or population of islets.

In some embodiments of this aspect of the disclosure, the pancreatic islet or population of islets is dissected from a pancreas or comprises cultured pancreatic islet cells.

Yet another aspect of the disclosure encompasses embodiments of an encapsulated cell aggregate, wherein the cell aggregate can be coated with a plurality of polymer bilayers, wherein each polymer bilayer comprises a poly(N-vinylpyrrolidone) (PVPON) polymer layer and a polyphenolic tannin layer, wherein the poly(N-vinylpyrrolidone) (PVPON) polymer layer and the polyphenolic tannin layer are hydrogen-bonded, and wherein at least one poly(N-vinylpyrrolidone) (PVPON) layer comprises a plurality of manganoporphyrin moieties conjugated to the poly(N-vinylpyrrolidone) (PVPON) layer, wherein the deposited plurality of polymer bilayers forms a capsule encapsulating the isolated cell or cell aggregate.

In some embodiments of this aspect of the disclosure, the cell aggregate can be an isolated pancreatic islet dissected from a pancreas or is a cultured pancreatic islet, and wherein the coated aggregate of cells is capable of producing insulin when the coated aggregate is transplanted into a recipient human or animal subject.

In some embodiments of this aspect of the disclosure, the capsule can further comprise a functional moiety attached thereto.

In some embodiments of this aspect of the disclosure, the functional moiety can be selected from the group consisting of: a detectable moiety, an immunomodulatory molecule, a growth factor, a cell receptor ligand, a polypeptide cell receptor, or any combination thereof.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 schematically illustrates the synthesis of carboxyl monofunctionalized tetrapyridyl Mn-porphyrin (MnP).

FIG. 2 illustrates the UV-vis spectra of carboxyl monofunctionalized tetrapyridyl porphyrin before (dotted) and after (solid) metalation with Mn (II). The spectra were taken from diluted aliquots of the aqueous metalation reaction (approximately 0.2 mg mL⁻¹).

FIG. 3A schematically illustrates the synthesis of tetrapyridyl Mn-porphyrin functionalized copolymer of poly(N-vinylpyrrolidone) (MnP-PVPON).

FIG. 3B illustrates the FTIR spectra of the PVPON copolymer before and after coupling to porphyrin.

FIG. 4A illustrates the UV-vis spectra of the PVPON-NH2 copolymer, MnP-PVPON, and a mixture of free MnP and PVPON-NH₂ after 96 h-dialysis in 0.01 M phosphate buffer at pH=7.4.

FIG. 4B illustrates the UV-vis spectrum of the MnP-PVPON solution at pH=7.4 (0.01 M) after being dialyzed for 1, 2, 5, 24, 48, and 120 h.

FIG. 5A schematically illustrates the multilayer assembly of tannic acid (TA), poly(N-vinylpyrrolidone (PVPON), and Mn-porphyrin functionalized PVPON (MnP-PVPON) on spherical sacrificial silica particles to obtain antioxidant porphyrin-polyphenolic hollow capsules.

FIG. 5B illustrates the multilayer architecture of the PVPON/TA hollow capsules with the porphyrin copolymer bilayer of MnP-PVPON/TA localized in the inner (S1, (MnP-PVPON/TA)₂(PVPON/TA)_{3.5}), middle (S2, (PVPON/TA)₂(MnP-PVPON/TA)₂(PVPON/TA)_{1.5}), or outer part (S3, (PVPON/TA)₄(MnP-PVPON/TA)_{1.5}) of the multilayer shell.

FIGS. 6A-6D illustrates SEM images of (FIGS. 6A, 6B) (PVPON/TA)₅ and (FIGS. 6C, 6D) (PVPON/TA)₂(MnP-PVPON/TA)₂(PVPON/TA) multilayer capsules assembled at pH=5 (0.01 M phosphate buffer) and dried on silicon wafers from deionized water solutions.

FIG. 6E is a graph illustrating the growth of TA/PVPON/MnP-PVPON multilayers at pH=5 and at pH=7.2 (0.01 M phosphate buffer) on silicon wafer surfaces as measured by in situ ellipsometry inside a 5-mL liquid cell.

FIGS. 6F-6G illustrate optical images of a water drop placed on (PVPON/TA)_{5.5} (FIG. 6F) and a (PVPON/TA)₄(MnP-PVPON/TA)_{1.5} (FIG. 6G) multilayers.

FIGS. 7A-7B are graphs illustrating the viability (%) of splenocytes from NOD mice after 48-h incubation with 2.5×10⁶ (FIG. 7A) and 5×10⁶ (FIG. 7B) capsules of (PVPON/TA)₅, (PVPON/TA)_{5.5}, (PVPON/TA)₂(MnP-PVPON/TA)₂(PVPON/TA) and (PVPON/TA)₂(MnP-PVPON/TA)₂(PVPON/TA)_{1.5} (labeled as (MnP-PVPON)* and (MnP-PVPON)**, respectively), as obtained from an MTT assay (absorbance was measured at 540 nm).

FIG. 8A illustrates a schematic for ABTS oxidation to produce ABTS⁺⁻ radical cation (left) and a catalytic cycle of metalloporphyrin reduction/oxidation (right).

FIG. 8B illustrates UV-vis spectra of ABTS⁺⁻ decolorization by (PVPON/TA)_{5.5} capsules.

FIG. 8C is a graph illustrating ABTS⁺⁻ discoloration by MnP-PVPON copolymer, (PVPON)6 hydrogels, (PVPON/TA)5.5, (MnP-PVPON/TA)₂(PVPON/TA)_{3.5}(S1), (PVPON/TA)₂(MnP-PVPON/TA)₂(PVPON/TA)_{1.5} (S2), and (PVPON/TA)₄(MnP-PVPON/TA)_{1.5} (S3) capsules.

FIG. 8D illustrates images of ABTS⁺⁻ solutions before (left) and after 20 min-treatment with (PVPON/TA)_{5.5} and S3 capsules.

FIG. 9A illustrates UV-vis spectra of the reduction of cytochrome C by superoxide.

FIG. 9B illustrates the results of a cytochrome C assay with (PVPON)₆ hydrogel capsules.

FIG. 9C illustrates the results of a cytochrome C assay for (PVPON/TA)_{5.5} and (PVPON/TA)₄(MnP-PVPON/TA)_{1.5} capsules in the presence of catalase.

FIGS. 10A-10D illustrate small-angle neutron scattering (SANS) data for (PVPON/TA)_{5.5} (FIGS. 10A, 10B) and (PVPON/TA)₄(MnP-PVPON/TA)_{1.5} (FIGS. 10C, 10D) capsules before (FIGS. 10A, 10C) and after (FIGS. 10B, 10D) ABTS⁺⁻ challenge. Solid lines represent model fits to SANS data.

FIG. 11 illustrates an ¹H NMR spectrum for compound 2 (*water in DMSO)

FIG. 12 illustrates an ¹H NMR spectrum for compound 3 (*water in DMSO)

FIG. 13 illustrates an ¹H NMR spectrum for compound 4.

FIG. 14 illustrates UV-vis spectra of ABTS+⁺ decolorization by (PVPON/TA)_{7.5}, (PVPON/TA)₆(MnP-PVPON/TA)_{1.5}, and (PVPON/TA)₄(MnP-PVPON/TA)_{3.5} capsules measured for 20 min at λ=734 nm.

FIG. 15 illustrates the results of a cytochrome C assay for (PVPON/TA)_{5.5} (labeled as PVPON/TA) and (PVPON/TA)₄(MnP-PVPON/TA)_{1.5} (labeled as MnP-PVPON/TA) capsules in the absence of catalase.

FIG. 16 illustrates a proposed mechanism of CAT-like activity of the porphyrin.

The drawings are described in greater detail in the description and examples below.

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

All average molecular weights of polymers are weight-average molecular weights, unless otherwise specified.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. Patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

### Abbreviations

LbL, layer-by-layer; MnP, manganoporphyrin; PVPON, poly(N-vinylpyrrolidone; TA, tannic acid; MnP-PVPON, manganoporphyrin-poly(N-vinylpyrrolidone) conjugate; TEM, transmission electron microscope; FITC, fluorescein isothiocyanate; ROX, reactive oxygen species.

### Definitions

In describing and claiming the disclosed subject matter, the following terminology will be used in accordance with the definitions set forth below.

The term “additional pharmacologically active agent” as used herein, refers to any agent, such as a drug, capable of having a physiologic effect (e.g., a therapeutic or prophylactic effect) on prokaryotic or eukaryotic cells, in vivo or in vitro, including, but without limitation, chemotherapeutics, toxins, radiotherapeutics, radiosensitizing agents, gene therapy vectors, antisense nucleic acid constructs or small interfering RNA, imaging agents, diagnostic agents, agents known to interact with an intracellular protein, polypeptides, and polynucleotides.

The additional pharmacologically active agent can be selected from a variety of known classes of drugs, including, for example, analgesics, anesthetics, anti-inflammatory agents, anthelmintics, anti-arrhythmic agents, antiasthma agents, antibiotics (including penicillins), anticancer agents (including Taxol), anticoagulants, antidepressants, antidiabetic agents, antiepileptics, antihistamines, antitussives, antihypertensive agents, antimuscarinic agents, antimycobacterial agents, antineoplastic agents, antioxidant agents, antipyretics, immunosuppressants, immunostimulants, antithyroid agents, antiviral agents, anxiolytic sedatives (hypnotics and neuroleptics), astringents, bacteriostatic agents, beta-adrenoceptor blocking agents, blood products and substitutes, bronchodilators, buffering agents, cardiac inotropic agents, chemotherapeutics, contrast media, corticosteroids, cough suppressants (expectorants and mucolytics), diagnostic agents, diagnostic imaging agents, diuretics, dopaminergics (antiparkinsonian agents), free radical scavenging agents, growth factors, haemostatics, immunological agents, lipid regulating agents, muscle relaxants, proteins, peptides and polypeptides, parasympathomimetics, parathyroid calcitonin and biphosphonates, prostaglandins, radio-pharmaceuticals, hormones, sex hormones (including steroids), time release binders, anti-allergic agents, stimulants and anoretics, steroids, sympathomimetics, thyroid agents, vaccines, vasodilators, and xanthines.

The additional pharmacologically active agent need not be a therapeutic agent. For example, the agent may be cytotoxic to the local cells to which it is delivered but have an overall beneficial effect on the subject. Further, the agent may be a diagnostic agent with no direct therapeutic activity per se, such as a contrast agent for bioimaging.

The terms “administering” and “administration” as used herein refer to introducing a composition of the present disclosure into a subject.

The term “aggregate” as applied to a “cell” herein refers to a plurality of a cell type or several cell types that may have been dissected (isolated) from a tissue, or have formed a multicellular body upon culturing in vitro. An exemplary aggregate isolated from an animal tissue is a pancreatic islet of Langerhans. Such an islet may include cells other than those identified as β-cells responsive to a stimulus such as glucose and which, in response thereto, synthesize into the surrounding medium insulin. Such an islet may also be formed in such as a liquid medium by culturing isolated pancreatic cells. Other cell aggregates for use in the compositions of the disclosure can include, but are not limited to, non-pancreatic cells that can produce hormones, cytokines, neuropeptides and the like that may be pathologically deficient in an animal or human subject.

The term “antibody” as used herein refers to polyclonal and monoclonal antibody preparations, as well as preparations including hybrid antibodies, altered antibodies, F(ab′)₂ fragments, F(ab) fragments, Fv fragments, single domain antibodies, chimeric antibodies, humanized antibodies, and functional fragments thereof which exhibit immunological binding properties of the parent antibody molecule.

The term “capsule” as used herein refers to a hollow structure wherein an internal volume is defined by an outer shell comprising a layer-by-layer composition according to the disclosure, wherein the layer-by-layer comprises bilayers consisting of PVPON and TA and wherein at least one PVPON has Mn-porphyrin moieties conjugated thereto. While the defined internal volume may be occupied by a core on which the layer-by-layer composition is formed, the internal volume may be voided on contents such as when the core former is removed, whereupon the volume may receive, for example an amount of a therapeutic agent.

The term “cell” as used herein refers to any natural or artificial cell, animal, plant, bacterial, or a viral particle that be viable or dead. Such cells may be isolated from an animal or human subject or tissue thereof, or a cultured cell previously isolated from a subject source. An artificial cell includes, but is not limited to, an artificially engineered entity derived from such as a unicellular microorganism wherein all or some of the genetic material has been replaced.

The term “coating” as used herein refers to a multilayered coating encapsulating a cell, an aggregate of cells, or a core structure such as, but not limited to a removable silica core, a nanoparticle, or the like. The coating may also be applied to a surface of other than a core such as, but not limited to, a substantially planar surface such as a silica wafer, and the like. In such a coating or coat of the present disclosure, a first layer or coat can comprise a polymer or units thereof that can be hydrogen-bonded to a substrate surface or to an outer cell membrane surface and, while thus bonded to a cell or cell aggregate does not significantly reduce the viability, physiology, or functioning of the cell type (for example, by retaining responsiveness to glucose in the case of coated pancreatic islets). In embodiments of the compositions of the disclosure the first layer can be, but is not limited to, poly(N-vinylpyrrolidone.

The term “functional moiety” as used herein refers to any molecule that may be attached to the outer surface of the outermost layer of the embodiments of the bilayer coatings of the disclosure. It is contemplated, but not intended to be limiting, for such moieties to be an imaging moiety (including a fluorescent dye, radiolabel, and the like), an immunomodulatory molecule, a growth factor, or any combination thereof, and the like.

The term “growth factor’ as used herein refers to a peptide or polypeptide that can be, but is not limited to, a ligand that specially binds to a polypeptide or other receptor of a cell and includes, but is not limited to, a Acrp30, adipocytes complement related protein 30 kDa (adiponectin); ALCAM, activated leukocyte cell adhesion molecule; BDNF, brain-derived neurotrophic factor; BLC, B-lymphocyte chemoattractant; BMP, bone morphogenetic protein; BTC, β-cellulin; CCR, CC-chemokine receptor; CLC, cardiotrophin-like cytokine; CV, coefficient of variance; CXCR, CXC-chemokine receptor; DAB, 3,3′-diaminobenzidine; DAN, differential screening-selected gene aberrative in neuroblastoma; ECL, enhanced chemiluminescence; EDG-1, estrogen down-regulated gene 1; EGF, epidermal growth factor; ELISA, enzyme-linked immunosorbant assay; ET-1, endothelin 1; ETAR, endothelin receptor type A; FGF, fibroblast growth factor; GDF, growth and differentiation factor; GFR, Glial cell line-derived neurotrophic factor receptor; HB-EGF, heparin-binding EGF-like factor; HCC, hemofiltrate CC chemokine; ICAM, intercellular adhesion molecule; IFN, interferon; IGF, insulin-like growth factor; IGFBP, insulin-like growth factor binding protein; IgG, immunoglobulin gamma; IL, interleukin; I-TAC, Interferon-inducible T-cell alpha chemoattractant; LCK, lymphocyte cell-specific protein-tyrosine kinase; LIF, leukemia inhibitory factor; MCP, monocytes chemoattractant protein; M-CSF, macrophage colony stimulating factor; MIP, macrophage inflammatory protein; MMP, matrix metalloproteinase; MSP, macrophage stimulating protein; NAP, neural antiproliferation factor; NGF, nerve growth factor; NRG, neuregulin; NT, neurotensin; PDGF, platelet-derived growth factor; PIGF, placental growth factor; SCF, stem cell factor; TARC, thymus- and activation-regulated chemokine; TGF, transforming growth factor; TIMP, tissue inhibitor of metalloproteinases; TNF, tumor necrosis factor; TNFRSF, TNF receptor superfamily member; TNFSF, TNF superfamily member; TRAIL, TNF-related apoptosis inducing ligand; TRANCE, tumor necrosis factor-related activation induced cytokine; uPAR, urokinase plasminogen activator receptor; VCAM, vascular cellular adhesion molecule; VEGF, vascular endothelial growth factor.

The term “imaging agent” as used herein refers to a labeling moiety that is useful for providing an indication of the position of the label and adherents thereto, in a cell or tissue of an animal or human subject, or a cell or tissue under in vitro conditions. Such agents may include those that provide detectable signals such as fluorescence, luminescence, radioactivity, or can be detected by such

The term “label” or “tag” as used herein refers to a molecule that, when appended by, for example, without limitation, covalent bonding or hybridization to another moiety, for example, also without limitation, a nanoparticle provides or enhances a means of detecting the other moiety. A fluorescence or fluorescent label or tag emits detectable light at a particular wavelength when excited at a different wavelength. A radiolabel or radioactive tag emits radioactive particles detectable with an instrument such as, without limitation, a scintillation counter. Other signal-generation detection methods include: chemiluminescence, electrochemiluminescence, raman, colorimetric, hybridization protection assay, and mass spectrometry. Radionuclides may be either therapeutic or diagnostic; diagnostic imaging using such nuclides is also well known. Typical diagnostic radionuclides include, but are not limited to, ⁹⁹Tc, ⁹⁵Tc, ¹¹¹In, ⁶²Cu, ⁶⁴Cu, ⁶⁷Ga, ⁶⁸Ga.

The term “porphyrin” as used herein refers to a group of heterocyclic macrocycle organic compounds, composed of four modified pyrrole subunits interconnected at their α carbon atoms via methine bridges (═CH—). With a total of 26 π-electrons, of which 18 π-electrons form a planar, continuous cycle, the porphyrin ring structure is often described as aromatic. One result of the large conjugated system is that porphyrins typically absorb strongly in the visible region of the electromagnetic spectrum, i.e. they are deeply colored. Macrocycle compounds with bridges of one carbon atom or one nitrogen atom respectively, joining pyrroles to form characteristic tetrapyrrole ring structure. There are many different classes of porphyrin-like compounds. The term porphyrins will be used herein to refer to porphyrins and metallo derivatives thereof.

Some porphyrins are isolated from nature, for example, protoporphyrin IX, which is the organic portion of hemin. Many derivatives of natural porphyrins are known (see, for example Smith & Cavaleiro (1986) Heterocycles 26:1947-1963). Other porphyrins can be synthesized in the laboratory including those made via the condensation of aldehydes and pyrroles, such as tetraphenylporphyrin. They also include compounds built up from smaller organic fragments.

Porphyrin-like compounds can have one or more substituents, and combinations of one or more different substituents. The substituents can be symmetrically or unsymmetrically located. The substituents, as well as the overall structure, can be neutral, positively charged or negatively charged. Charged structures have counterions. Metals can be inserted into the tetrapyrrole ring. including, but are not limited to, Fe, Co, Zn, Mo, Ti, Mn, Cr, Ni, Mg, Cu, Tl, In, Ru, V and Au. In the embodiments of the disclosure a preferred metal is manganese to form a manganoporphyrin (MnP).

The term “multilayered composition” as used herein refers to a layer-by-layer-formed structure of superimposed polymer layers. The layers can be alternating PVPON and TA layers that bond by hydrogen bonds. In the coatings of the disclosure, at least one of the PVPON layers is modified by having manganese-porphyrin (MnP) moieties conjugated thereto. In some embodiments, the MnPs moieties are conjugated by coupling PVPON-NH₂ copolymer with a carboxyl mono-functionalized tetrapyridyl Mn-porphyrin via carbodiimide chemistry to form an MnP-PVPON layer. In some embodiments, the MnP-PVPON layer can be embedded within the multilayered composition, thereby having a TA layer on each side of the MnP-PVPON layer, or a PVPON on one side and a TA layer on the opposing side. In other embodiments, the MnP-PVPON layer is disposed on one surface of the PVPON-TA bilayer.

The term “nanoparticle” as used herein refers to a particle having a diameter of between about 1 and about 1000 nm, preferably between about 100 nm and 1000 nm, and most preferably between about 50 nm and 700 nm. Similarly, by the term “nanoparticles” is meant a plurality of particles having an average diameter of between about 50 and about 1000 nm. The term “nanoparticle” as used herein may refer to a core component encapsulated by a layer-by layer coating according to the disclosure or to a capsule formed from an MP-PVPON-TA layer-by layer coating composition of the disclosure.

The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to a diluent, adjuvant, excipient, or vehicle with which a layer-by layer capsule of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the layer-by layer capsule and pharmaceutically acceptable carriers can be sterile. Water is a useful carrier when the layer-by layer capsule is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use.

The pharmaceutical compositions of the subject invention can be formulated according to known methods for preparing pharmaceutically useful compositions. Formulations containing pharmaceutically acceptable carriers are described in a number of sources which are well known and readily available to those skilled in the art. For example, Remington's Pharmaceutical Sciences (Martin E W, Remington's Pharmaceutical Sciences, Easton Pa., Mack Publishing Company, 19.sup.th ed., 1995) describes formulations that can be used in connection with the subject invention. Formulations suitable for parenteral administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

The term “pharmacologically active agent” as used herein, refers to any agent, such as a drug, capable of having a physiologic effect (e.g., a therapeutic or prophylactic effect) on eukaryotic cells, in vivo or in vitro, including, but without limitation, chemotherapeutics, toxins, radiotherapeutics, radiosensitizing agents, gene therapy vectors, antisense nucleic acid constructs or small interfering RNA, imaging agents, diagnostic agents, agents known to interact with an intracellular protein, polypeptides including but not limited to, antibodies, and polynucleotides, and a biologic such as oncolytic viruses.

The term “polymer” as used herein refers to molecules comprising two or more monomer subunits that may be identical repeating subunits or different repeating subunits. A monomer generally comprises a simple structure, low-molecular weight molecule containing carbon. Polymers may optionally be substituted. A preferred polymer of the disclosure is polyvinylpyrrolidone.

The term “polymer bilayer” as used herein refers to a first layer of poly(N-vinylpyrrolidone) and a layer of a polyphenol (tannic acid) hydrogen-bonded thereto. In embodiments where the bilayers encapsulate a cell or aggregate of cells, it is preferred that the layer being proximal to the underlying cell or cells is poly(N-vinylpyrrolidone). In such embodiments, the outermost biocompatible layer, not having a polyphenol layer thereon, may be derivatized for the attachment of such as a labeling moiety, or other functional moiety. The coatings of the disclosure further include at least one poly(N-vinylpyrrolidone) layer wherein some or all of the poly(N-vinylpyrrolidone) units have conjugated thereon manganophorphyrin moieties. The resulting poly(manganoporphyrin-N-vinylpyrrolidone) (MnP-PVPON) layer(s) may be located as the inner most layer of the capsule structure that is proximal to an encapsulated volume or in contact with a surface such as, but not limited to, a cell membrane, sandwiched within non-manganoporphyrin-containing bilayers, or as the outermost layer most distal from the encapsulated volume or bilayer-covered surface. It is also contemplated that if the MnP-PVPON layer is the outermost layer of a composition according to the disclosure then the PVPON units of the polymer layer may be further modified by the attachment thereto of other functional moieties as herein disclosed.

The term “polyphenol” as used herein refers to structural class of natural, synthetic and semi-synthetic organic chemicals characterized by the presence of large multiples of phenol units generally moderately water-soluble compounds, with molecular weight of 500-4000 Da, at least 12 phenolic hydroxyl groups, and 5-7 aromatic rings per 1000 Da, where the limits to these ranges are necessarily somewhat flexible, and include, but are not limited to the tannins.

The term “(PVPON/TA)ₙPVPON” as used herein refers to a multi-layered composition such as, but not limited to a coating of a silica surface, a cell, or to plurality of cells according to the present disclosure, the coating comprising “n” layers. The designator “n” denotes the number of bilayers on the multi-layered coating, “n” ranging from at least one to about 10. In embodiments where “n” is 1.5, 2.5, 3.5, 4.5, and the like, the 0.5 denotes that the multi-layered coating has an outer layer of poly(N-vinylpyrrolidone) not having a polyphenol (e.g. tannic acid) layer disposed thereon.

The term “reactive oxygen species” (ROX) as used herein are chemically reactive chemical species containing oxygen. Examples include peroxides, superoxide, hydroxyl radical, singlet oxygen, and alpha-oxygen. In a biological context, ROS are formed as a natural byproduct of the normal metabolism of oxygen and have important roles in cell signaling and homeostasis. However, during times of environmental stress (e.g., UV or heat exposure), ROS levels can increase dramatically. This may result in significant damage to cell structures.

The term “subject” or “patient” as used herein means both mammals and non-mammals. Mammals include, for example, humans; non-human primates, e.g. apes and monkeys; and non-primates, e.g. dogs, cats, cattle, horses, sheep, and goats.

The term “support surface” as used herein refers a surface receiving a layer-by-layer composition according to the disclosure. In some embodiments, the support surface is that of a silica core that may be removed from the layer-by-layer construct to leave a volume or space encapsulated by a capsule. In some other embodiments, the support surface can be a substantially planar surface such as, but not limited to a silica or glass wafer on which the layer-by-layer composition of the disclosure is deposited.

The terms “treating” or “treatment” as used herein refer to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects.

The term “volume” as used herein refers to a space that is defined by a layer-by-layer capsule. The layer of a polymer bilayer is closest to the volume or the contents contained therein is the “proximal” layer whereas the polymer layer the furthest from the volume (space) or the contents contained therein is the “distal” layer.

### Description

The present disclosure provides embodiments of a synergistic manganoporphyrin-polyphenol polymeric nano-thin coatings and hollow microcapsules comprising at least one such manganoporphyrin-polyphenol polymeric layer having antioxidant activity and controllable ROS modulation. These embodiments are produced by multilayer assembly of a polyphenolic antioxidant, tannic acid (TA), with a copolymer of polyvinylpyrrolidone comprising conjugated manganoporphyrin moieties (MnP-PVPON), synthesized by coupling a PVPON-NH₂ copolymer with a carboxyl monofunctionalized tetrapyridyl Mn-porphyrin via carbodiimide chemistry, that mimics the enzymatic antioxidant superoxide dismutase. The redox activity of the copolymer increases the antioxidant response of MnP-PVPON/TA capsules compared to unmodified PVPON/TA capsules not having manganoporphyrin moieties and suppresses the proliferation of superoxide via cytochrome C competition.

MnP-PVPON copolymer is stable in aqueous solutions at physiological pH with no change in its manganese coordination. MnP-PVPON can be successfully assembled with TA with the bilayer thickness of (MnP-PVPON/TA) assembled at pH=5 and is smaller than that at pH=7.4 (0.9±0.1 nm versus 1.8±0.1 nm, respectively), which is the counter-trend to MnP-free (PVPON/TA) assembly.

The MnP-PVPON/TA multilayer capsules exhibit a highly hydrophilic nature and is found to be non-toxic to NOD splenocytes after as much as a 48-h incubation. Inclusion of MnP-PVPON as an outer layer enhances radical scavenging activity as compared to locating the MnP-PVPON layer in the middle or inner part of a capsule shell or bilayered coating of a surface. In addition, TA contributes to the synergistic radical scavenging activity of the MnP-PVPON/TA system that exhibits a combined superoxide dismutase-like ability and catalase-like activity in response to the free radical superoxide challenge.

The MnP-PVPON/TA containing capsules of the disclosure exhibit a low degree of the loss of shell thickness upon free radical treatment, while PVPON/TA capsules can lose as much as about 40% of their shell thickness due to the non-catalytic free radical scavenging of TA, as demonstrated by Small Angle Neutron Scattering (SANS). The manganoporphyrin-polyphenol capsules have been found to be non-toxic to splenocytes obtained from NOD.BDC-6.9 mice after 48-h incubation. Accordingly, the combination of catalytic activity of manganoporphyrins with natural polyphenolic antioxidants provides free radical scavenging materials that can be useful in antioxidant therapies, for the formation of viable encapsulated cells, and as free-radical dissipating protective carriers of biomolecules for biomedical or industrial applications.

The present disclosure further encompasses the use of MnP-conjugated polymer layers in nanoscale coatings and methods of manufacture thereof suitable for coating isolated cells or aggregates of cells. The coatings can be attached to the cells by hydrogen-bonding to proteins on the surface of the cells or to extracellular matrix proteins such as collagen. The coatings of the disclosure, using a hydrogen-bonded, layer-by-layer approach, can be particularly advantageous for application to cell aggregates useful for transplantation therapy such as, but not limited to, individual pancreatic islets of Langerhans by providing islets suitable for transplantation with the possibility of prolonged viability and physiological functioning compared to other methods of preparation of islets. It is considered within the scope of the disclosure, however, that the coatings of the disclosure may be used for the preparation of any cell type or group of cells that could usefully provide such as hormonal products, neurotransmitters or neuroregulators, physiological modulators and the like that may be down-regulated or pathologically absent from a human or animal. Besides providing insulin for the treatment of diabetic patients, for example, cells coated by the compositions and methods of the disclosure can be used to provide dopamine for the control of the symptoms of Parkinson's disease, and the like.

Accordingly, the disclosure provides embodiments of cytoprotective coatings comprising, through hydrogen-bonded interactions, polyphenol (TA) and poly(N-vinylpyrrolidone) (PVPON) deposited on a group of cells via Layer-by-layer (LbL) assembly and including at least one MnP-PVPON layer. This approach allows for the introduction of material, such as the tannic acid, that is able to modulate adaptive immune responses, crucial for cell enhanced viability and prolonged function after transplantation into a recipient animal or human and further comprises the protective effect against reactive oxygen species provided by the MnP-PVPON polymer layer(s). In contrast to the currently available approaches based on LbL, the compositions and methods of the disclosure (a) involve non-cationic non-toxic compounds; (b) the compounds can be modified before deposition onto islet surfaces. The coatings are conformal over various types of cell aggregates.

This approach utilizes conformational hydrogel-like coatings based on hydrogen-bonded interactions of natural polyphenols (tannic acid; TA) with poly (N-vinylpyrrolidone) (PVPON). This protocol is simple, fast (5-10 min), and non-toxic (hydrogen-bonding at neutral pH for protein assembly), involves biocompatible materials with antioxidant properties (tannic acid contains biologic activity as an antioxidant, antimicrobial, anti-carcinogenic, anti-mutagenic, and antibacterial agent competent to reduce free radical-induced oxidation of adjacent molecules), is comprised of permeable, ultrathin (less than 100 nm) coatings of minimal void volume, allows for coating of individual islets, and is adaptable to direct functionalization by forming complexes with molecules such as imaging agents, catalytic antioxidants (e.g., CA), and/or immunomodulatory molecules (e.g., FasL). Most significantly, the PVPON layer can be modified by conjugating thereto manganoporphyrin moieties that can synergistically cooperate with a TA layer to reduce the level of ROS. Coating thickness can be easily controlled at the nanoscale level by varying the number of polymer levels. Coating porosity/permeability is controlled by the chemistry of the assembled polymers.

Thus, the present disclosure provides methods of synthesis of an ultrathin bio-mimetic coating that can be applied, for example, to any type of cell including, but not limited to animal cells, bacterial cells, and the like, that allow the compositions of the disclosure to be attached to the underlying cellular substrate by hydrogen bonding. In one particularly useful application of the methods and compositions of the present disclosure, the coatings and methods of the disclosure can also coat aggregates of cells such as, but not limited to, isolated or cultured islets of Langerhans for pancreatic islet modification.

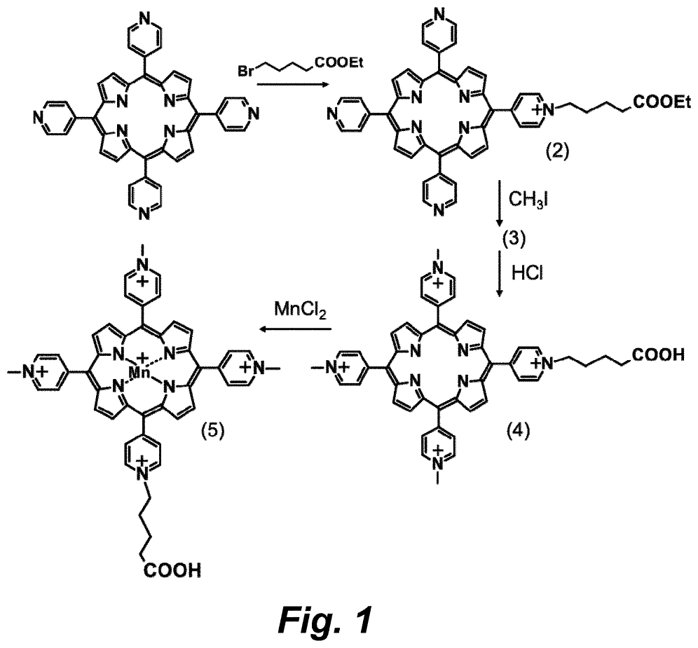

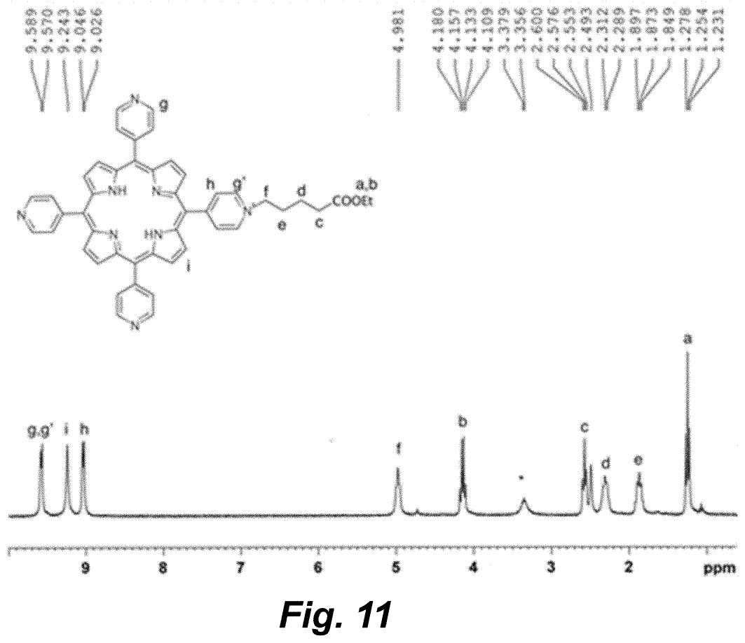

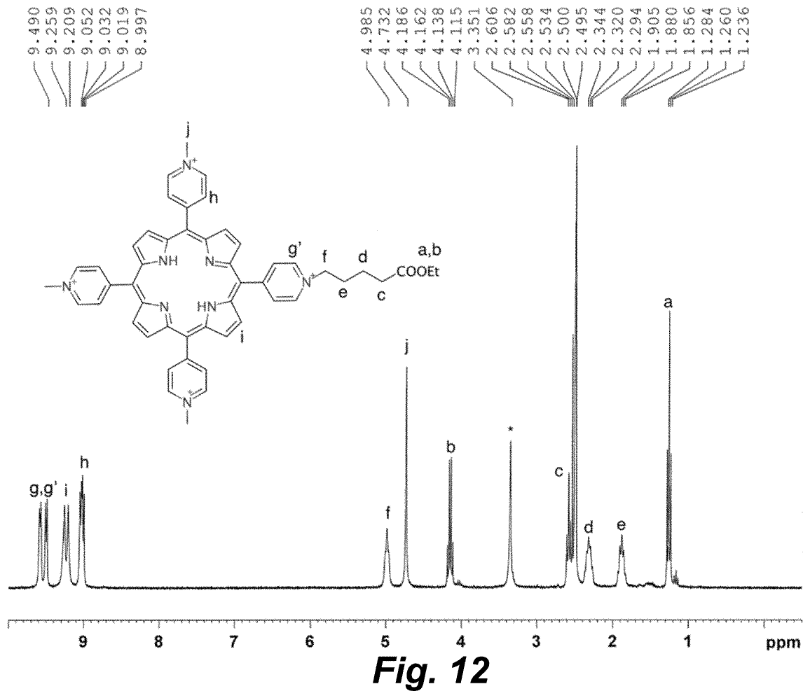

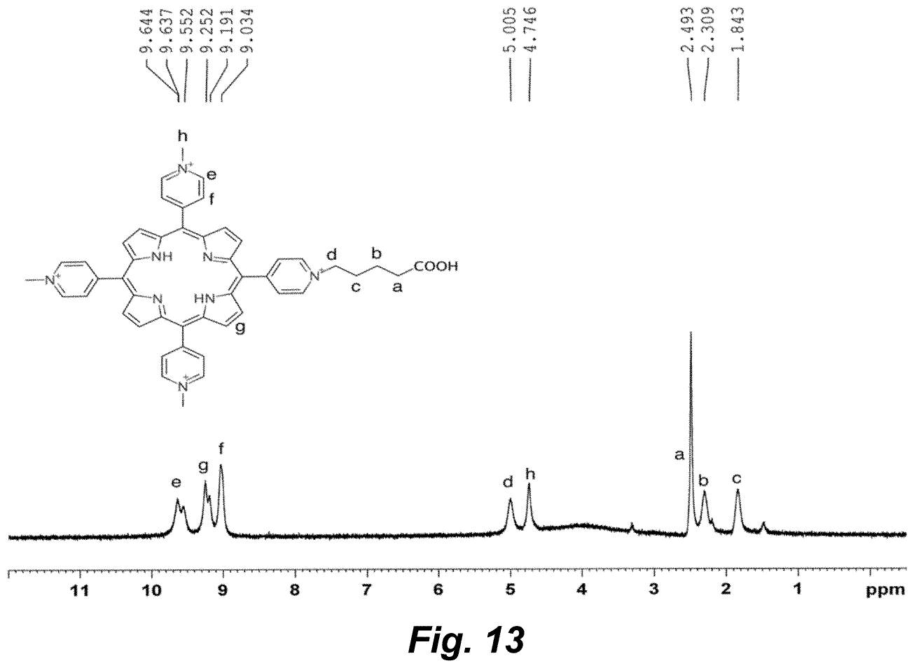

Synthesis of the Mn(II) porphyrin-PVPON copolymer: The carboxyl monofunctionalized tetrapyridyl Mn-porphyrin (MnP) was synthesized via monoquaternization of meso-tetra (4-pyridyl) porphine with bromopentanoate, followed by methylation of the remaining pyridyl groups with methyl iodide and hydrolysis of the ester function to obtain meso-tetrakis-(1-methylpyridinium-4-yl) porphyrin monopentaacetic acid (FIG. 1; compounds 1-4). The ¹H NMR analysis of 2-4 confirmed the presence of the oxoethyl proton signals at 4.27 ppm (2H, —OCH₂CH₃) and 0.6-1 ppm (3H, —OCH₂CH₃) before and after methylation and the appearance of the proton signals at 4.8 ppm (from methyl groups after methyl iodide reaction (FIGS. 11 and 12) (Asayama et al., (2004) Bioconjugate Chem. 15: 1360-1363). The disappearance of the oxoethyl proton signals also was confirmed during the complete hydrolysis of the ester group (FIG. 13).

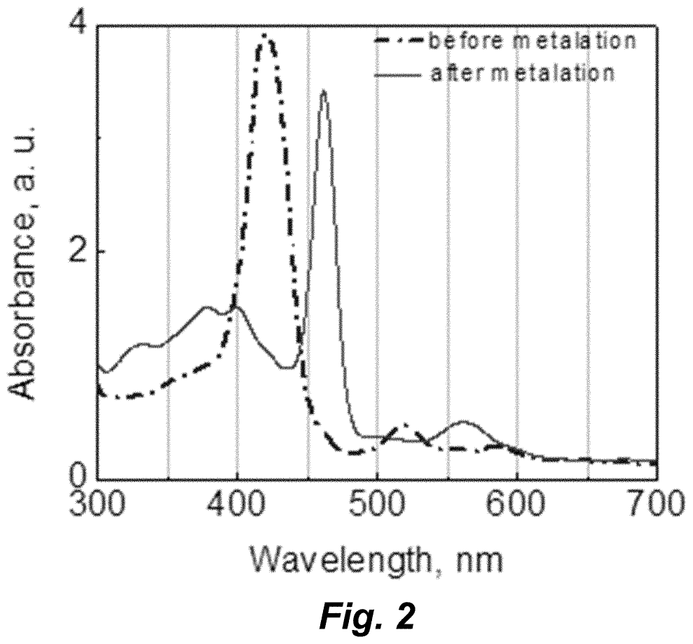

The metalation of manganese (III) meso-tetrakis-(1-methylpyridinium-4-yl) porphyrin pentaacetic acid (FIG. 1; 5) was performed by reflux with manganese (II) chloride in deionized water for 80 min. The UV-vis spectroscopy analysis in FIG. 2 shows that the Soret band of the porphyrin shifted from 420 nm to 462 nm after introduction of manganese ions into the porphyrin core (Asayama et al., (2004) Bioconjugate Chem. 15: 1360-1363).

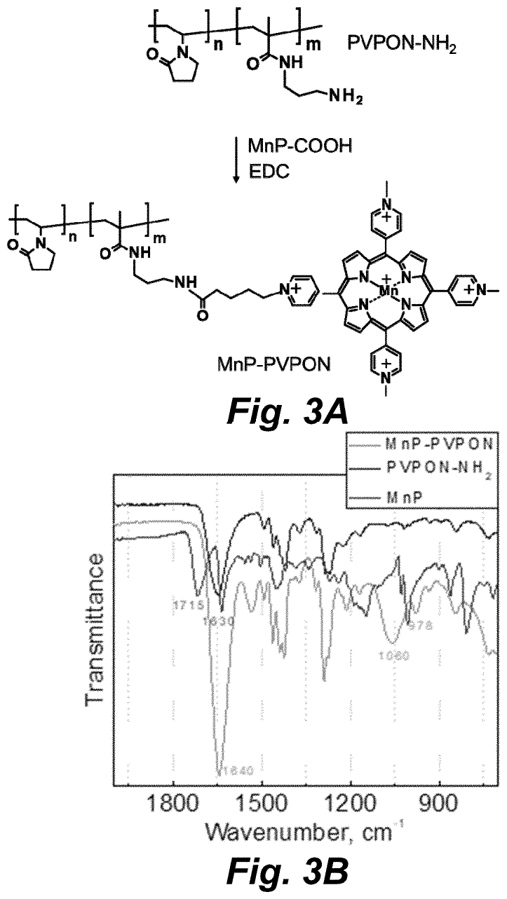

To obtain the SOD mimicking PVPON, the Mn-porphyrin was covalently conjugated to the PVPON-NH₂ copolymer via carbodiimide-assisted reaction of the free amine groups on the methyl aminopropylacrylamide units of the copolymer and the pentanoic acid function of the porphyrin (FIG. 3A). The 20% MnP-PVPON was selected as it is a high NH₂-containing molar percentage that still results in stable (TA/MnP-PVPON) multilayer growth. The MnP-polymer was purified from the unreacted MnP by extensive dialysis for 96 hours. FTIR analysis revealed the disappearance of the carboxylic acid of the porphyrin observed at 1715 cm⁻¹ due to the amide bond formation (FIG. 3b). The amide band was combined with the PVPON amide at 1640 cm⁻¹ in the obtained MnP-PVPON copolymer. The new peaks in the MnP-PVPON copolymer around 1060 and 978 cm⁻¹ are due to the characteristic porphyrin peaks (Saeedi et al., (2013) Polyhedron 49, 158-166) that shifted from 1174 and 1004 cm⁻¹ to lower frequencies due to steric quenching (Minaev & Lindgren (2009) Sensors 9: 1937-1966) from the surrounding polymer chain (FIG. 3B).

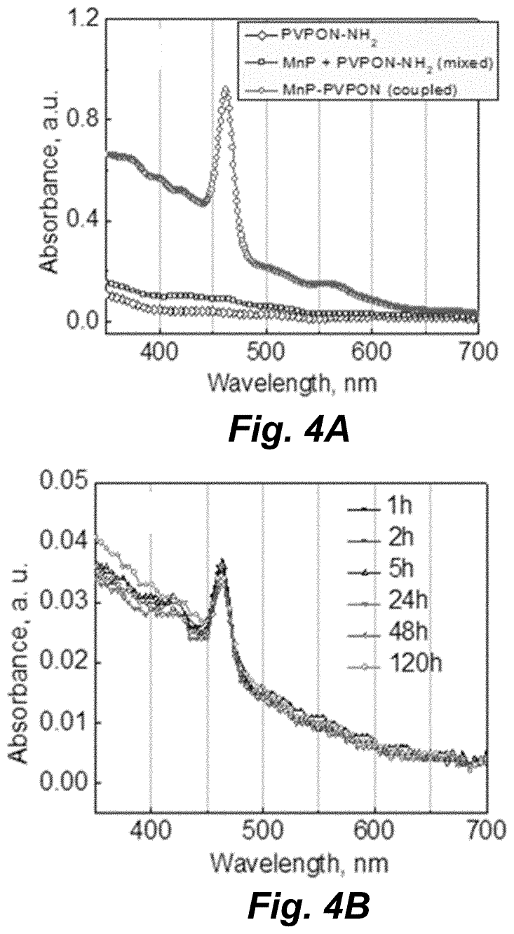

To additionally confirm the covalent modification of PVPON-NH₂ with MnP versus non-covalent hydrophobic or polar interactions between the copolymer and MnP molecules, UV-vis spectrophotometric analysis of a solution of PVPON-NH₂ mixed MnP in the absence of EDC followed by 96-h dialysis in water was conducted. FIG. 4A shows that in contrast to the characteristic Soret band of the Mn (III) porphyrin in the spectrum of MnP-PVPON at 464 nm, the spectra of PVPON-NH₂ and extensively dialyzed PVPON-NH2 mixture with MnP (FIG. 4A, MnP-PVPON (mixed)) presented no major absorbance bands in the visible spectrum. The stability of the porphyrin attachment to the PVPON copolymer and the manganese entrapment in the porphyrin core was examined using UV-vis spectroscopy of aqueous solutions of (1 mg mL⁻¹) MnP-PVPON solutions at pH=5 and pH=7.4.

The overlaid UV-vis spectra in FIG. 4B demonstrate that no change in the Soret band absorbance occurred after dialysis of the MnP-PVPON solution at pH=7.4 for 120 h. Since the Soret band significantly shifts towards lower energies during metalation of the porphyrin (FIG. 2), any loss of manganese ions would result in the appearance of a Soret band at the original wavelength of 422 nm which was not observed during the dialysis (FIG. 4B). Similarly, no decrease of the band absorbance after MnP-PVPON dialysis confirmed retention of the porphyrin pendant functionalities during extended time in aqueous solution. These results showed that the PVPON-NH₂ copolymer did not produce noncovalent assemblies with the porphyrin in aqueous solutions and all free porphyrin molecules could be removed via water dialysis.

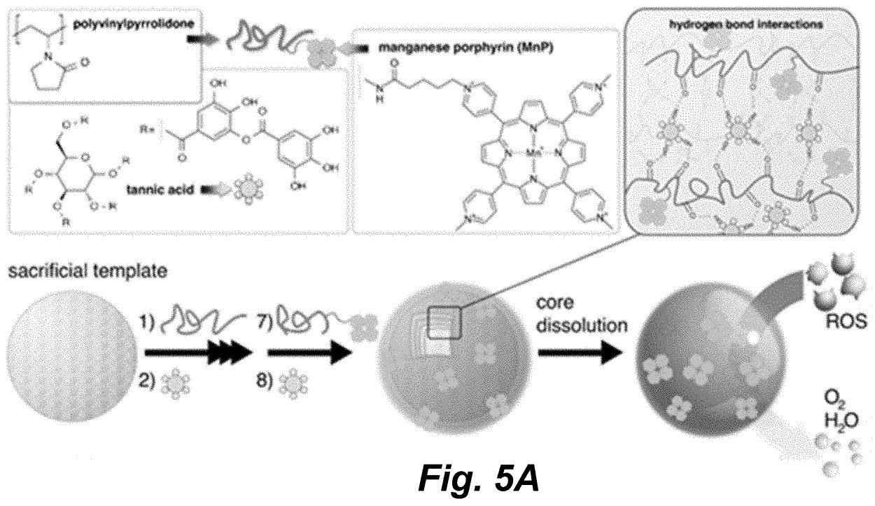

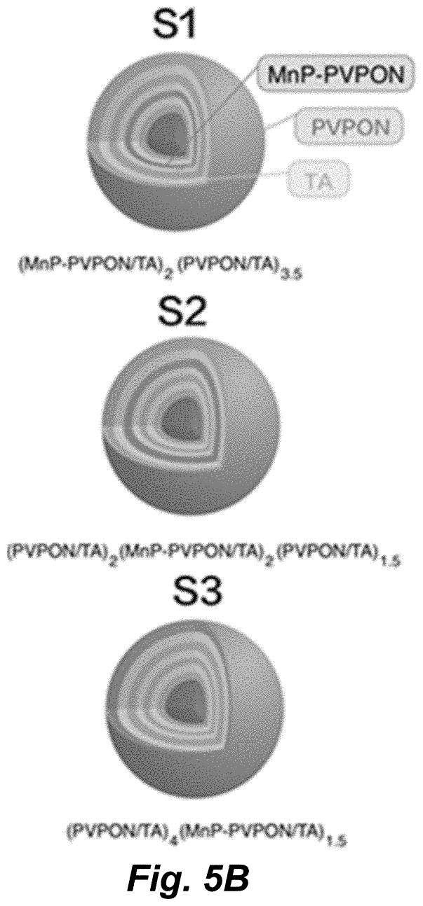

Multilayer assemblies of the MnP-PVPON with TA and their cytocompatibility: The capability of MnP-PVPON to assemble with TA at pH=5 and pH=7.4 into multilayer coatings on silica surfaces was studied. FIG. 5A schematically shows that multilayer assembly between TA phenolic groups and MnP-PVPON carbonyls occurs through hydrogen bonding (Liu et al., (2014) Soft Matter 10: 9237-9247). However, there was a possibility that cationic MnP moieties on PVPON chains could inhibit multilayer assembly of TA and MnP-PVPON due to an increase of the ratio of positive to negative charges within the multilayer and as a result to mutual repulsions between pyridinium positive charges on MnP (Kharlampieva & Sukhishvili (2003) Langmuir 19: 1235-1243).

To test the capability of MnP-PVPON to assemble with TA, three types of hollow multilayer capsules 4 μm in diameter were synthesized with the shell architecture and denoted as S1, S2, and S3, where the porphyrin copolymer bilayer of MnP-PVPON/TA was localized in the inner (MnP-PVPON/TA)₂(PVPON/TA)_{3.5}, middle (PVPON/TA)₂(MnP-PVPON/TA)₂(PVPON/TA)_{1.5}, or outer part (PVPON/TA)₄(MnP-PVPON/TA)_{1.5} of the multilayer shell, respectively (FIG. 5B) using multilayer deposition of the polymers from 0.01 M phosphate buffer solutions at pH=5.

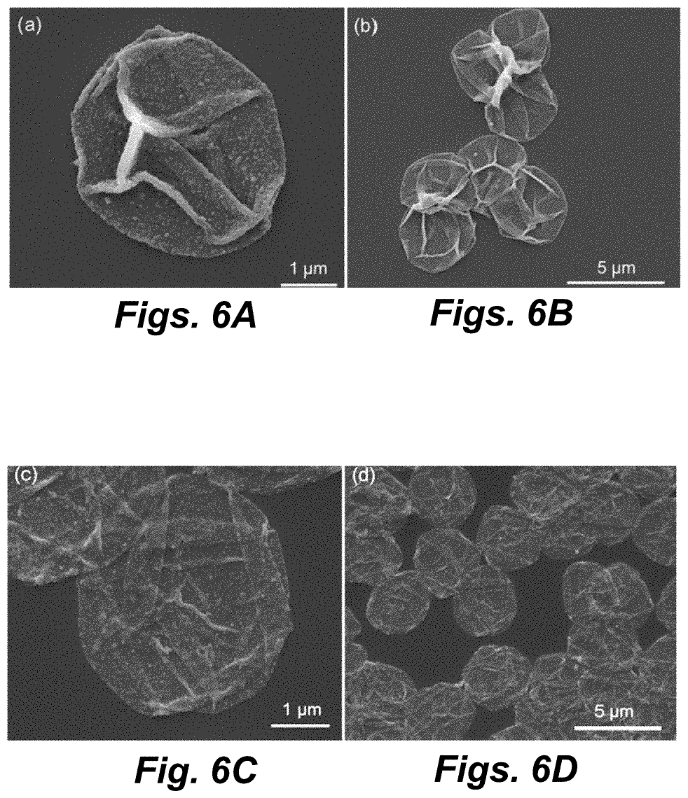

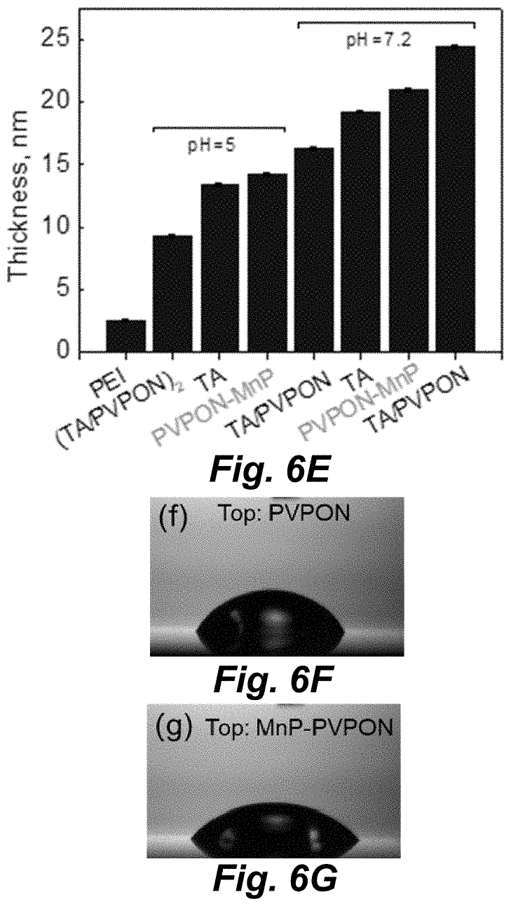

The SEM analysis of the (PVPON/TA)₅ (FIGS. 6A and 6B) and (PVPON/TA)₂(MnP-PVPON/TA)₂(PVPON/TA) capsules (FIGS. 6C and 6D) with five total bilayers of (PVPON/TA) after core dissolution and dialysis revealed that the MnP-PVPON-containing capsules were much thinner than their (PVPON/TA) counterparts, in agreement with the ellipsometry results at the same deposition pH=5 (FIG. 6E). In contrast to (PVPON/TA)₅ capsules which collapse upon drying leaving protruding thick folds and rigid creases, the porphyrin-polyphenolic capsules were fully collapsed demonstrating minimal wrinkling, a typical feature of soft multilayer capsules (FIG. 6a-d) (Kozlovskaya et al., (2009) Chem. Mater. 21: 2158-2167). Similarly, the increased shell thickness of (PVPON/TA) capsules was demonstrated to result in increased rigidity of the capsule shell which could support their three-dimensional spherical structure upon drying (Kozlovskaya et al., (2012) Adv. Funct. Mater. 22: 3389-3398; Gao et al., (2001) Eur. Phys. J. E 5: 21-27).

Since SEM morphology analysis of dry polymer multilayers can be ambiguous due to possible artifacts, e.g., increased roughness, loss of material, or drying, the SEM results were rationalized by studying the TA/MnP-PVPON multilayer growth at slightly acidic and neutral pH values using in situ ellipsometry. FIG. 6E shows in situ thicknesses of the polymer layers adsorbed from polymer solution at pH=5 followed by that at pH=7.2 (0.01 M phosphate buffer) on silicon wafer surfaces as measured inside a 5-mL liquid cell without intermediate drying, i.e. wet thickness. We found that while MnP-PVPON can be successfully adsorbed on top of the TA layer in both cases, the bilayer thickness of (TA/MnP-PVPON) assembled at pH=5 is smaller than that at pH=7.4. The (TA/MnP-PVPON) bilayer at pH=7.4 resulted in 1.8±0.1 nm, a two-fold larger growth than that at pH=5, which gave only 0.9±0.1 nm (TA/MnP-PVPON) bilayer thickness. In contrast, the opposite thickness trend was observed for porphyrin-free TA/PVPON growth, where increasing the deposition pH value from pH=5 to pH=7.4 led to a 2-fold decrease in capsule thickness with 4.2 nm and 1.3 nm per a bilayer for pH=5 (0.01 M) and pH=7.4 (0.01 M), respectively (Liu et al., (2014) Soft Matter 10: 9237-9247).

The pH-dependent difference in the (TA/PVPON) bilayer thickness was likely due to unshielded ionization of TA at high deposition pH that resulted in mutual repulsion of the neighboring layers of TA and slightly thinner coatings at pH=7.4 (Liu et al., (2014) Soft Matter 10: 9237-9247). This effect has been reported for other multilayer systems: for instance, glutaraldehyde-assisted growth of cationic PAH or poly(L-lysine) occurred only at high salt conditions (0.1 M) that allowed the screening of similar positive charges on adjacent polyelectrolyte chains (Tong et al., (2006) Macromol. Rapid Commun. 27: 2078-2083; Wang et al., (2008) Colloids Surf. A 329: 58-66).

The results indicate that at low 0.01 M salt conditions, the MnP-PVPON interactions with TA at pH greater than 5 can involve both hydrogen bonding and ionic pairing due to increased TA ionization that should also allow 38% thicker PVPON/TA coatings when MnP-PVPON is used compared to that of (PVPON/TA) when MnP-free PVPON is employed during the multilayer deposition. The stability of the MnP-PVPON/TA multilayers dried from pH=2.5 was examined since ionization of TA is minimized at this pH (below the pKₐ of TA), which could result in destabilization of the film. The MnP-PVPON/TA bilayer did not show any significant thickness changes after exposure to pH=2.5 as compared to the film stability at pH=5 (Table 1).

TABLE 1
Ellipsometric measurements on dry films of TA/MnP-PVPON
at the pH of assembly (5) and below the pKa of tannic acid (2.5).

| | | Total | SD | Layer |
|---|---|---|---|---|
| pH 5 | Layer | thickness (nm) | (nm) | thickness (nm) |
| | SiO₂ | 2.3 | 0.1 | |
| | TA | 4.1 | 0.2 | 1.8 |
| | MnP-PVPON | 6.8 | 0.2 | 2.7 |
| pH 2.5 | MnP-PVPON | 6.4 | 0.2 | 2.3 |

Cationic pendant MnP moieties on the PVPON backbone resulted in a decreased hydrophobicity of the coating as observed from sessile drop contact angle measurements using water as a contact liquid. The contact angle of a water drop decreased from 61°±0.1° to 49°±0.2° when the last layers in (PVPON/TA)_{5.5} were changed to (MnP-PVPON/TA)_{1.5} (FIGS. 6F and 6G).

The observed increased wettability of the (MnP-PVPON)_{1.5} surface compared to (PVPON/TA)_{1.5} as a top stack is probably due to the high hydrophilicity of the porphyrin moiety that implies useful of the system in biomedically relevant environment, as hydrophilic polymers prevent negative immune response and rapid body clearance (Seong & Matzinger (2004) Nat. Immunology 4: 469-478; Moyano et al., (2012) J. Am. Chem. Soc. 134: 3965-3967; Gaucher et al., (2009) Biomacromolecules 10: 408-416; Andersen et al., (2011) Biomaterials 32: 4481-4488).

Despite the hydrophilic nature of TA/MnP-PVPON, there was the possibility of cytotoxicity due to cationic pyridinium groups (Huang et al., (2016) Chem. Sci. 7: 7013-7019) and manganese cations (Stephenson et al., (2013) Toxicol. Lett. 218: 299-307; Park et al., (2017) Biol. Pharm. Bull. 40: 1275-1281) of the MnP pendant groups that can be cytotoxic if released from PVPON. To examine the potential cytotoxic effects of TA/MnP-PVPON, an MTT assay was performed to assess the viability of splenocytes from NOD mice, a spontaneous mouse model of Type 1 diabetes (Anderson & Bluestone (2005) Annu. Rev. Immunol. 23: 447-485) following a 48-h incubation with (PVPON/TA)₂(MnP-PVPON/TA)₂(PVPON/TA) and (PVPON/TA)₂(MnP-PVPON/TA)₂(PVPON/TA)_{1.5} capsules, with (PVPON/TA)₅, and (PVPON/TA)_{5.5} as negative cytotoxicity controls (Pham-Hua et al., (2017) Biomaterials 128: 19-32).

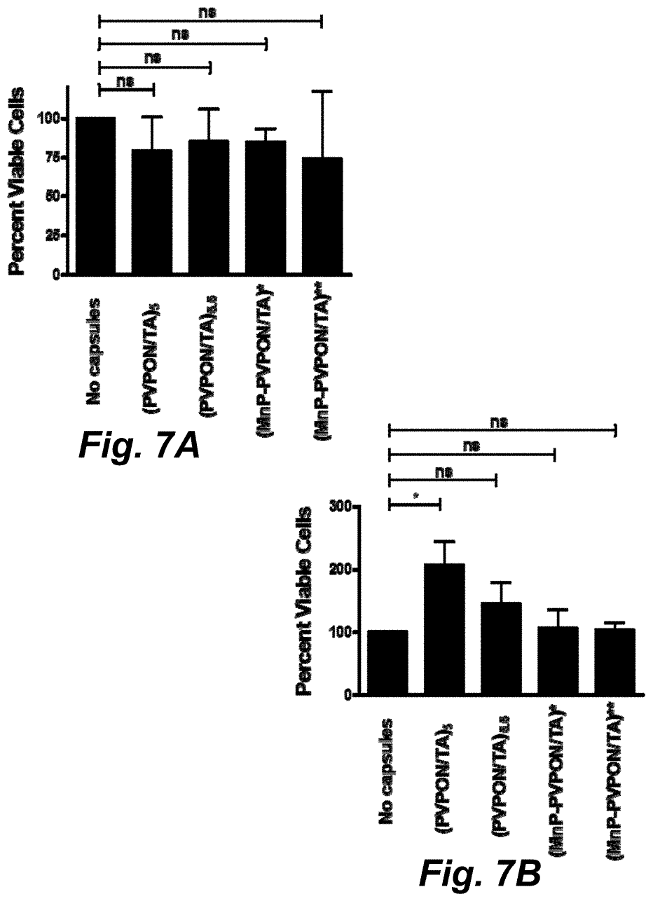

Examining the potential cytotoxic effects of TA/MnP-PVPON on immune cells was essential for defining the feasibility of this material for future in vivo studies. FIG. 7A displays non-cytotoxic MnP-PVPON-containing capsules at the concentration of 2.5×10⁶ μL⁻¹ and percent viability from formazan, a product of intracellular enzyme cleavage of the initial MTT, that are similar to a non-treated control and both negative cytotoxicity controls. The two-fold increased capsule concentration of 5×10⁶ μL⁻¹ did not induce cell cytotoxicity from MnP-PVPON-containing capsules (FIG. 7b) further demonstrating the stability of the porphyrin-polyphenolic system in vitro.

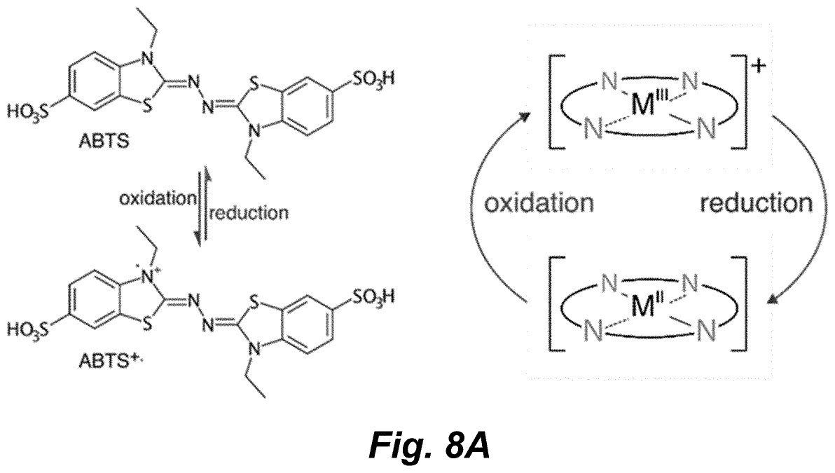

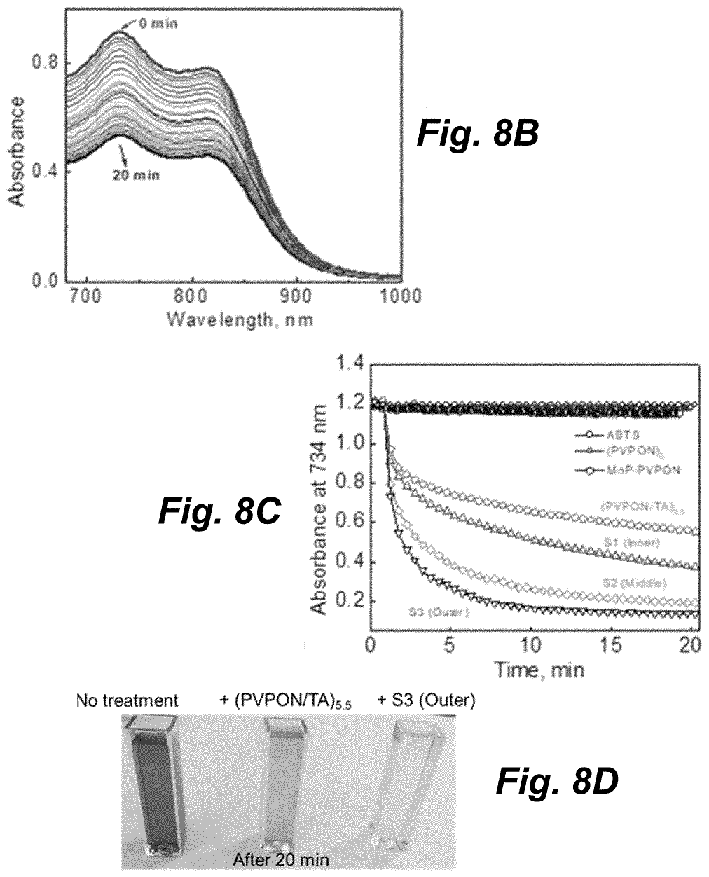

Antioxidant and free radical scavenging properties of metalloporphyrin-polyphenolic multilayers: The presence of MnP functionalities within (PVPON/TA) capsules can change their radical scavenging ability was examined by measuring the kinetics of their interaction with 2,2′-azinobis(3-ethylbenzothiazoline-6-sulfonic acid) diammonium salt (ABTS⁺) radical cation. When ABTS is oxidized to the stable radical (FIG. 8A), an absorbance at 734 nm from a brilliant blue-green solution of the ABTS⁺⁻ can be used to monitor its decolorization upon reduction by an electron donor (FIG. 8B). Inclusion of the MnP-PVPON copolymer dramatically enhanced the antioxidant ability of the materials.

FIG. 8c shows that in the first 1-2 minutes, quick ABTS⁺⁻ decolorization occurred in the presence of (PVPON/TA)_{5.5} capsules followed by a slower decomposition of ABTS⁺⁻ for the remaining 18 mins, in agreement with other findings on free radical scavenging activity of the (PVPON/TA) capsules as measured by luminol oxidation assay. The ability of TA to preserve its antioxidant properties being ionically paired into a multilayer with various polycations also has been demonstrated previously (Shutova et al., (2007) Russ. J. Gen. Chem. 77: 1494-1501).

An enhanced response to the ABTS⁺⁻ radical cation challenge was observed when two PVPON layers were exchanged for MnP-PVPON in the outer (PVPON/TA)_{1.5} stack. The optical density of the ABTS⁺⁻ solution decreased in the presence of (PVPON/TA)₄(MnP-PVPON/TA)_{1.5} capsules by 4-fold more than that by (PVPON/TA)_{5.5} capsules in 20 min indicating a reduction of 4-fold more radical cation species during the same time. The images of ABTS⁺⁻ solutions before and after 20 min-treatment with (PVPON/TA)_{5.5} and (PVPON/TA)₄(MnP-PVPON/TA)_{1.5} (labeled as S3, FIG. 5B) capsules demonstrate that a complete loss of ABTS⁺⁻ color is observed in the presence of S3 capsules while a significant but incomplete decolorization of ABTS⁺⁻ solution occurred in the case of (PVPON/TA)_{5.5} capsules (FIG. 8D).

The location of the MnP-PVPON layers within the 5.5-bilayer capsule shell was examined. Positioning the porphyrin stack away from the outer surface slowed down the ABTS⁺ decolorization and decreased the radical scavenging ability of the system (the number of the radical cation species being reduced) (FIG. 8C). Thus, for instance, the initial ABTS⁺⁻ optical density decreased 8.5-fold by S3 (outer) capsules, and 6- and 3.2-fold by S2 (middle) and S1 (inner) capsules (FIG. 5B), respectively, while it was reduced only by 2.1-fold when MnP-PVPON-free capsules of (PVPON/TA)_{5.5} were used (FIG. 8C). ABTS⁺⁻ radical cations can diffuse through a different environment and are being reduced by different species in the case of (PVPON/TA)_{5.5} and S1, S2, and S3 capsules. In the case of (PVPON/TA)_{5.5} and S1 and S2, the outer PVPON likely partially shields TA from the radical cations due to hydrogen bonds between the phenolic groups in TA and the carbonyls in PVPON. These bonds need to be broken to reduce the radical, which slows the rate of the radical cation reduction (Shutova et al., (2007) Russ. J. Gen. Chem. 77: 1494-1501). In contrast, in S3 capsules the outer stack contains active MnP groups and is able to immediately reduce the incoming ABTS⁺⁻ radical cations.

The control (PVPON)₆ hydrogel capsules and MnP-PVPON polymer did not demonstrate any significant change to the ABTS⁺⁻ concentration during the test time of 20 mins (FIG. 8C). This result confirms the absence of PVPON antioxidant activity as demonstrated previously using the luminol oxidation assay (Kozlovskaya et al (2015) Adv. Healthcare Mater. 4: 686-694) and provides some insight into the synergistic relationship between TA and the MnP-PVPON for the radical scavenging activity of (TA/MnP-PVPON) capsules. As reported, manganese porphyrins have manganese ions in the 3⁺ oxidation state (Haber & Gross (2015) Chem. Commun. 51: 5812-5827). A single one-electron MnP catalytic loop reduces the manganese to the 2⁺ oxidation state by accepting an electron followed by an oxidation to the 3⁺ oxidation state again (FIG. 8A) (Haber & Gross (2015) Chem. Commun. 51: 5812-5827). However, decolorization of the ABTS⁺⁻ radical occurs through the reduction of the radical to ABTS in the ground state (FIG. 8A) (Branchi et al., (2005) Org. Biomol. Chem. 3: 2604-2614). The half-cell reduction potential of the opposite step, i.e., further oxidation of ABTS⁺⁻ to ABTS⁺⁺ is 1.1 V, which is higher than the oxidation-limited catalysis of Mn(II)Cl₂ (Branchi et al., (2005) Org. Biomol. Chem. 3: 2604-2614; Batinic-Haberle et al., (2006) Dalton Transact., 617-624) and did not appear to occur during the assay (characterized by an increase in absorbance near 565 nm).

Since no absorbance shift was seen over time in the presence of the free MnP-PVPON polymer, we suggest that MnP-PVPON on its own may not assist redox reactions where the first step is oxidation of the material, and therefore TA is required to activate the catalytic MnP loop. In this case the close proximity of TA phenoxy radicals formed through initial oxidation by ABTS⁺⁻ may allow the porphyrin to participate in catalytic cycles where ABTS⁺⁻ is reduced alongside formation and disproportionation of peroxides as manganoporphyrins have been shown to exhibit some catalase-like activity (Day et al., (1997) Arch. Biochem. Biophys. 347: 256-262; Milano & Day (2000) Nucleic Acids Research 28: 968-973). From the shape of the curves in the ABTS reaction it is apparent that the reduction mechanism is complex, with the initial sharp decrease followed by a slow, steady decline indicative of multiple steps. Importantly, since the thickness of the multilayer was shown to be decreased by inclusion of the MnP-PVPON at the assembly pH=5, the increased antioxidant activity per capsule can be attributed to inclusion of the manganoporphyrin within the capsule shell.

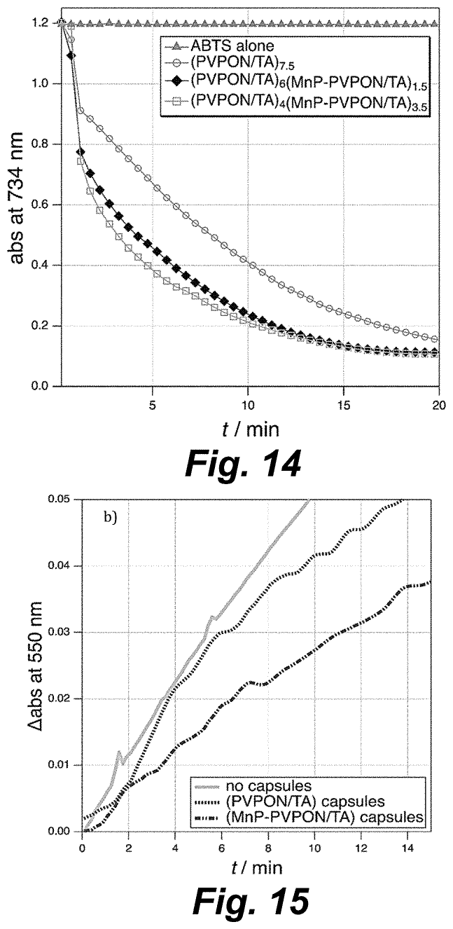

Increasing the number of MnP-PVPON bilayers instead of PVPON layers was not necessary to further increase the rate of ABTS⁺⁻ reduction and incorporation of the MnP-PVPON polymer as the outer two layers of the capsule shell was sufficient to greatly enhance the radical scavenging ability of the (PVPON/TA) capsules. Thus, for instance, substitution of two additional MnP-PVPON layers into (PVPON/TA)₆(MnP-PVPON/TA)_{1.5} capsules to obtain (PVPON/TA)₄(MnP-PVPON/TA)_{3.5} resulted in similar radical scavenging abilities of the MnP-containing systems. In the higher layer capsules, the discoloration occurred 2-times quicker with (PVPON/TA)₄(MnP-PVPON/TA)_{3.5} capsules than when (PVPON/TA)_{7.5} was used with both showing a plateau of effectiveness after 10 min in the ABTS⁺⁻ assay (FIG. 14).

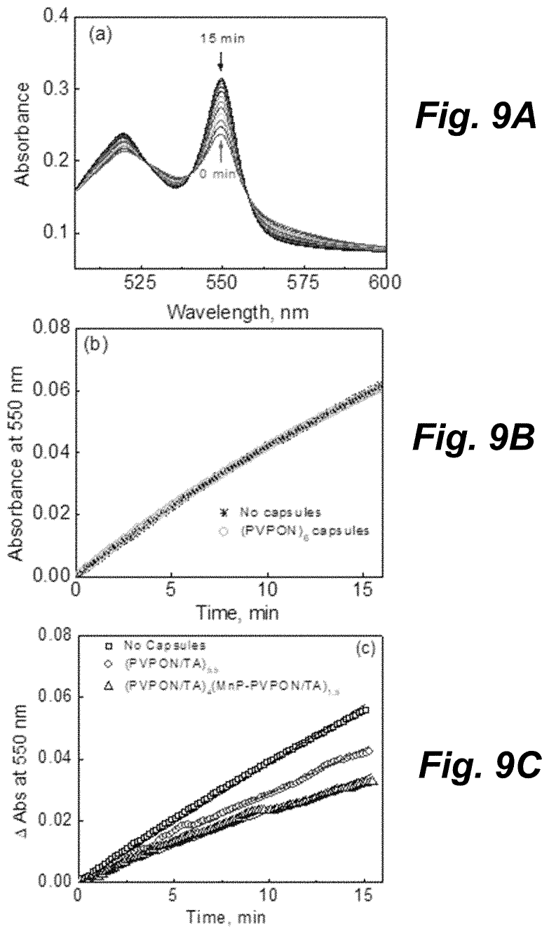

Superoxide dismutation by manganoporphyrin-polyphenolic capsules: The ability of the MnP-PVPON/TA system to dismute superoxide radicals was studied by using a cytochrome C competition assay. The xanthine/xanthine oxidase reaction was used as a source of superoxide radicals which, upon reduction of cytochrome C in solution, can be traced by a steadily increasing absorbance at 550 nm (FIG. 9A). The rate of superoxide dismutation by MnP-PVPON/TA capsules was evaluated in comparison with (PVPON)₆ hydrogel capsules and MnP-free (PVPON/TA)_{5.5} capsules in the presence of catalase to prevent re-oxidation of cytochrome C by hydrogen peroxide, which is a major product of O₂⁻ dismutation (Vandewalle & Petersen (1997) FEBS Lett. 210: 195-198).

FIG. 9B shows that no reduction in the observed rate of superoxide radical production from the hydrogel capsules was observed, which agrees with the absence of activity from (PVPON)₆ capsules in scavenging ABTS⁺⁻ radical cations (FIG. 8C). In contrast, a significant lowering of the rate of cytochrome C reduction is observed in the presence of (PVPON/TA)_{5.5} capsules (FIG. 9B) due to the antioxidant properties of TA. Using the average literature value of Δε (reduced-oxidized) of cytochrome C at 550 nm (Sawada & Yamazaki (1973) Biochim. Biophys. Acta-Enzymology 327: 257-265; Van Gelder & Slater (1962) Biochim. Biophys. Acta 58: 593-595)=20.3 mM⁻¹ cm⁻¹, and the slope of the linear fit to absorbance change in FIG. 9C, the rate of superoxide production in the control experiment (no capsules were added) was found to be 189 nM min⁻¹. The MnP-free capsules were found to scavenge the superoxide radical at a rate of 47 nM min⁻¹ (189 nM min⁻¹ minus the observed rate of 142 nM min⁻¹ in the presence of 1×10⁷ (PVPON/TA)_{5.5} capsules). Remarkably, the same concentration of porphyrin-containing (PVPON/TA)₄ (MnP-PVPON/TA)_{1.5} capsules increased the superoxide scavenging rate to 77 nM min⁻¹ which represents a 64% increase in the radical scavenging efficiency per capsule for the MnP-containing capsules over their porphyrin-free predecessors and can be seen as a marked decrease in the fitted slope (FIG. 9C).

The results on superoxide dismutation by MnP-PVPON/TA capsules are in agreement with the SOD-like activity reported for various free porphyrins. It is worth noting that without catalase to scavenge H₂O₂ produced during O₂⁻⁻ dismutation, the rate of superoxide reduction by MnP-PVPON-containing capsules was still linear, unlike the (PVPON/TA)_{5.5} capsules that produced kinetic curves with uneven gradation of the slope (FIG. 15). Importantly, this observed difference can indicate the combined ability of the MnP-PVPON/TA system to convert both superoxide to H₂O₂ (superoxide dismutase-like ability) and H₂O₂ to water (catalase-like activity).

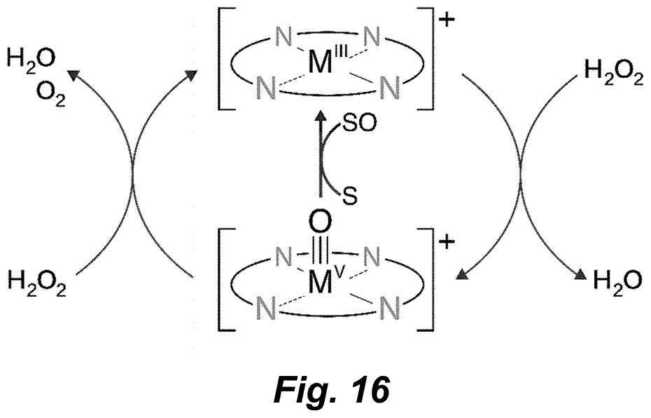

Manganoporphyrins have been previously reported to catalytically consume H₂O₂ and protect endothelial cells against the damaging effects of hydrogen peroxide based on the mechanism similar to catalase. The ring conjugation was speculated to be essential in delocalizing charge for the redox active metal and providing the stability of the manganic porphyrins in oxidative environments (FIG. 16). In contrast to that, TA scavenges H₂O₂ non-catalytically and may be slowly polymerized during the process (Shutava et al., (2004) Biomacromolecules 5: 914-921).

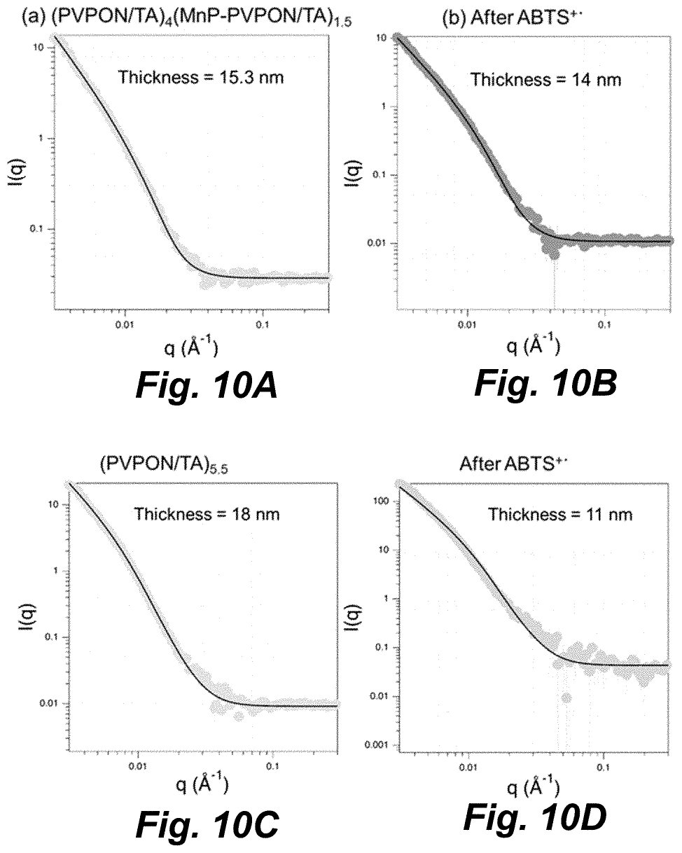

To probe the effect of the free radical challenges on the shell material of MnP-free and MnP-containing (PVPON/TA) multilayer capsules, we employed small angle neutron scattering (SANS) was used and evaluated the thickness of the capsule shell in solution before and after ABTS⁺⁻ radical cation treatment. The SANS data was fitted to a lamellar model (FIGS. 10A-10D) and the shell thickness was extracted from model parameters as has been studied previously in SANS studies of polyelectrolyte capsules in solution using the core-shell model (Estrela-Lopis et al., (2007) Langmuir 23: 7209-7215; Estrela-Lopis et al., (2009) Soft Matter 5: 214-219).

The 5.5-bilayer (PVPON/TA)₄(MnP-PVPON/TA)_{1.5} capsules were thinner than their MnP-free (PVPON/TA)_{5.5} counterparts with the capsule shell thicknesses of 15.3 and 18 nm, respectively, in agreement with the SEM and in situ ellipsometry results (FIGS. 6A-6D and FIG. 6E, respectively) and with the previously reported thickness of (PVPON/TA) capsules assembled at pH=5 (Kozlovskaya et al., (2010) Soft Matter 6: 3596-3608; Lisunova et al., (2011) Langmuir 27: 11157-11165). These (PVPON/TA) thickness results show that the MnP-PVPON/TA bilayer thickness fits to 2.8 nm (in contrast to 3.3 nm per PVPON/TA bilayer), is in agreement with the thinner MnP-PVPON/TA bilayers as measured by in-situ ellipsometry (FIG. 8E).

The moderate expansion of thickness may be attributed to the reduced ability of D₂O to screen charges and the reduced hydrogen bond-driven solvation in D₂O due to the higher energy required to disrupt the O-D bond network (Gittings et al., 2000) J. Phys. Chem. B 104 (18), 4381-4386). The importance of the thinner MnP-PVPON/TA shell is the implication that the antioxidant activity of the MnP-PVPON capsules can be attributed more to the porphyrin moiety, since TA represents a reduced compositional percentage in (PVPON/TA)₄(MnP-PVPON/TA)_{1.5} capsules compared to the (PVPON/TA) capsules. Despite the limited TA inclusion MnP-containing capsules still exhibit drastically enhanced ROS scavenging activity (FIGS. 8C and 9C).

Together with the synergistic relationship between TA and MnP in oxidative environments, inclusion of the manganoporphyrin polymer had a protective effect on the integrity of the capsule shell. For example, when the same capsules from the initial SANS scattering experiments were challenged with the ABTS⁺⁻ radical, the thickness of the (PVPON/TA)₄(MnP-PVPON/TA)_{1.5} capsule shell decreased by only 8% of the initial value with the after-treatment thickness of 14 nm (FIGS. 10A and 10B). In contrast, the MnP-free shell of (PVPON/TA)_{5.5} capsules lost 39% of the initial thickness resulting in a thickness of 11 nm after the radical challenge (FIGS. 10C and 10D). Apparently, the ability of the manganoporphyrin to disproportionate radicals catalytically drastically increased the stability of the PVPON/TA capsule shell by minimizing material loss due to degradation of TA in the oxidative environment. In the case of the MnP-free shell, the radicals are likely absorbed through conversion of phenol hydrogens to stabilized phenoxy radicals (Ingold & Howard (1962) Nature 195: 280) upon conversion of the phenols in TA into quinones which would result in slow partial disruption of hydrogen bonds between polyphenol molecules and the carbonyls of PVPON. The observed loss of the (PVPON/TA) shell thickness can therefore be attributed to this H-bond disruption.

The hydrogen-bonded Layer-by-Layer (LbL) technology of the disclosure can be usefully applied to coat living cells or aggregates of cells. The coatings of the disclosure are (a) conformal with uniform coverage over the whole cell aggregate surface; (b) stable for an extended period; (c) non-toxic; (d) can be easily modified with functional molecules, such as the fluorescent label FITC; and (e) can be applied to cell aggregates isolated from a range of animal species such as, but not limited to, rat, non-human primate, and human.

The thickness of the shell can be controlled by changing the molecular weight of PVPON or by increasing the strength of association between the polyphenol and a polymer. For instance, TA/PVPON planar LbL films assembled from PVPON with Mw=55 000 Da and with Mw=1 300 000 Da led to an increased bilayer thickness. The respective bilayer thickness values are 1.0±0.1 nm and 2.2±0.2 nm for the shells fabricated on silica cores. This allows varying the bilayer thickness in the coating of the same chemical composition without changing a number of deposited polymer pairs. In contrast, for ionically assembled (PLL-graft-PEG/alginate) LbL shells used for islet coating, it is necessary to change the deposition conditions, e.g., pH or ionic strength, to achieve the similar effect thus undermining the stability of the coating.

Coatings of controlled porosity and permeability necessary for regulating access of molecules towards, for example, encapsulated cell aggregates can be enhanced by functionalization of the (PVPON-TA)ₙ coating surface through such as PEGylation and biotinylation due to introduced amine groups mimicking islet surface chemistry. Copolymers of PVPON containing amine groups (the functionalization degree varied in the range of 5-20%) (PVPON-co-NH₂) were synthesized. PEGylation of PVPON-co-NH₂ was carried out through grafting of NHS-PEG chains to amine groups on PVPON backbone to produce PVPON-g-PEG. The grafted PEG were unbranched, hydrophilic, discrete-length molecules in the form of methyl-PEGₙ-NHS ester, where the subscript “n” denotes 4, 8, 12, or 24 ethylene glycol units. The N-hydroxysuccinimide (NHS) ester end group is spontaneously reactive with primary amines (—NH₂), providing for efficient PEGylation of amine-containing molecules or surfaces. Then, the (PVPON-TA)₃ coating was produced around islets according to the procedures of the disclosure and a layer of PVPON-g-PEG was deposited on top. For immobilization of immunomodulating, anti-coagulating or apoptotic ligand, grafting of sulfo-NHS-Biotin or sulfo-NHS-LC-Biotin (Pierce Biotechnology, Inc) to PVPON-co-NH₂ was performed to produce functional PVPON-g-Biotin that could be deposited as a last layer onto (PVPON-TA)ₙ-coated islets. The successful grafting of PEG molecules to PVPON was confirmed by gel permeation chromatography.

Bis-succinimide ester-activated PEG molecules can be used for grafting to PVPON-co-NH₂, to visualize the PEG-terminated coating in confocal microscopy. The N-hydroxysuccinimide ester (NHS) groups at one end of the PEG5 or PEG9 spacer react specifically and efficiently with amino groups on PVPON-coNH₂ at pH 7-9 to form stable amide bonds while the ester group at the other end will be fluorescently labeled with Alexa Fluor 488 carboxylic acid hydrazide sodium salt.

One aspect of the present disclosure, therefore, encompasses embodiments of a multilayered composition comprising a plurality of polymer bilayers, wherein the polymer bilayers each comprises a poly(N-vinylpyrrolidone) (PVPON) polymer layer and a polyphenolic tannin layer, wherein the poly(N-vinylpyrrolidone) (PVPON) polymer layer and the polyphenolic tannin layer are hydrogen-bonded, and wherein at least one poly(N-vinylpyrrolidone) (PVPON) layer comprises a plurality of manganoporphyrin moieties conjugated to the poly(N-vinylpyrrolidone) (PVPON) layer.

In some embodiments of this aspect of the disclosure, the multilayered composition is configured as a multi-layered capsule defining a volume or is disposed as a multilayered coating on a support surface.

In some embodiments of this aspect of the disclosure, the multilayered composition can comprise between about 3 polymer bilayers and about 10 polymer bilayers.

In some embodiments of this aspect of the disclosure, the polymer layer proximal to the defined volume or proximal to the support surface can be a poly(N-vinylpyrrolidone) (PVPON) layer.

In some embodiments of this aspect of the disclosure, at least one poly(N-vinylpyrrolidone) (PVPON) layer is embedded within the multilayered composition and comprises a plurality of manganoporphyrin moieties conjugated thereto.

In some embodiments of this aspect of the disclosure, the multilayered composition further comprises a poly(N-vinylpyrrolidone) (PVPON) layer disposed on the plurality of polymer bilayers as a layer most distal from the defined encapsulated volume or from the support surface and further comprising a plurality of manganoporphyrin moieties conjugated thereto.

In some embodiments of this aspect of the disclosure, the multilayered composition can further comprise an animal cell or aggregate of animal cells within the volume defined by the multi-layered capsule.

In some embodiments of this aspect of the disclosure, the polymer layer of the multi-layered capsule proximal to the defined volume of the capsule is hydrogen bonded to the cell membrane of a cell or a plurality of cells within the volume defined by the multi-layered capsule.

In some embodiments of this aspect of the disclosure, at least one polymer layer of the multilayered composition can further comprise a functional moiety attached thereto, and wherein the functional moiety can be selected from the group consisting of: a detectable moiety, an immunomodulatory molecule, a growth factor, or a combination thereof, and wherein the at least one polymer layer optionally comprises a molecular group suitable for attaching the functional moiety to the polymer layer.

In some embodiments of this aspect of the disclosure, the multi-layered capsule can further comprise a pharmacologically active compound that reacts with a reactive oxygen species when delivered to a recipient animal or human subject.

In some embodiments of this aspect of the disclosure, the compound that reacts with a reactive oxygen species can be a pharmacologically active agent.

In some embodiments of this aspect of the disclosure, the multilayered composition can be admixed with a pharmaceutically acceptable carrier.

Another aspect of the disclosure encompasses embodiments of a method of encapsulating an isolated cell or cell aggregate, the method comprising the steps of: (a) providing an isolated cell or cell aggregate; and (b) encapsulating the isolated cell or cell aggregate by depositing a plurality of polymer bilayers, wherein each polymer bilayer comprises a poly(N-vinylpyrrolidone) (PVPON) polymer layer and a polyphenolic tannin layer, wherein the poly(N-vinylpyrrolidone) (PVPON) polymer layer and the polyphenolic tannin layer are hydrogen-bonded, and wherein at least one poly(N-vinylpyrrolidone) (PVPON) layer comprises a plurality of manganoporphyrin moieties conjugated to the poly(N-vinylpyrrolidone) (PVPON) layer, wherein the deposited plurality of polymer bilayers can form a capsule encapsulating the isolated cell or cell aggregate.

In some embodiments of this aspect of the disclosure, the polymer layer in contact with the isolated cell or cell aggregate is poly(N-vinylpyrrolidone) hydrogen-bonded to the outer surface of the cell or aggregate of cells, or to an extracellular matrix component of the cell aggregate.

In some embodiments of this aspect of the disclosure, the method can further comprise the step of attaching at least one functional moiety to the capsule encapsulating the cells or cell aggregate.

In some embodiments of this aspect of the disclosure, the functional moiety can be selected from the group consisting of: a detectable moiety, an immunomodulatory molecule, a growth factor, a cell receptor ligand, a polypeptide cell receptor, or any combination thereof.

In some embodiments of this aspect of the disclosure, the cell or cell aggregate can be selected from the group consisting of: a bacterial cell, a viral cell, a plant cell, an artificial cell, and an animal cell, and wherein the cell or aggregate of cells is an isolated cell or aggregate of cells or a cultured cell or aggregate of cells.

In some embodiments of this aspect of the disclosure, the cell aggregate can be an isolated human or animal pancreatic islet or population of islets.

In some embodiments of this aspect of the disclosure, the pancreatic islet or population of islets is dissected from a pancreas or comprises cultured pancreatic islet cells.

Yet another aspect of the disclosure encompasses embodiments of an encapsulated cell aggregate, wherein the cell aggregate can be coated with a plurality of polymer bilayers, wherein each polymer bilayer comprises a poly(N-vinylpyrrolidone) (PVPON) polymer layer and a polyphenolic tannin layer, wherein the poly(N-vinylpyrrolidone) (PVPON) polymer layer and the polyphenolic tannin layer are hydrogen-bonded, and wherein at least one poly(N-vinylpyrrolidone) (PVPON) layer comprises a plurality of manganoporphyrin moieties conjugated to the poly(N-vinylpyrrolidone) (PVPON) layer, wherein the deposited plurality of polymer bilayers forms a capsule encapsulating the isolated cell or cell aggregate.

In some embodiments of this aspect of the disclosure, the cell aggregate can be an isolated pancreatic islet dissected from a pancreas or is a cultured pancreatic islet, and wherein the coated aggregate of cells is capable of producing insulin when the coated aggregate is transplanted into a recipient human or animal subject.

In some embodiments of this aspect of the disclosure, the capsule can further comprise a functional moiety attached thereto.

In some embodiments of this aspect of the disclosure, the functional moiety can be selected from the group consisting of: a detectable moiety, an immunomodulatory molecule, a growth factor, a cell receptor ligand, a polypeptide cell receptor, or any combination thereof.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified.

### EXAMPLES

### Example 1

Materials: N-vinylpyrrolidone (VPON), poly(N-vinylpyrrolidone) (PVPON, Mw=1,300,000 Da), mono- and dibasic sodium phosphate, ethyl-1-bromopentanoate, tetrahydrofuran (THF), tannic acid (TA, MW=1700 Da), petroleum ether, diethyl ether, methanol, isopropanol, dioxane, trolox, 2,2-azino-bis(3-ethylbenzothiazoline-6-sulfonic acid) diammonium salt (ABTS), potassium persulfate, cytochrome C, xanthine, xanthine oxidase, ethylene diamine tetraacetic acid (EDTA), and catalase were purchased from Fisher Scientific and used without further purification except where otherwise mentioned. N-(t-BOC-aminopropyl)methacrylamide (tBOC) was purchased from Polysciences. Solid silica cores were purchased from Cospheric. 3-(3-dimethylaminopropyl)-1-ethyl-carbodiimide hydrochloride (EDC) was purchased from Chem-Impex International. Ultrapure deionized water with a resistivity of 18.2 Ω cm was used in all experiments (Evoqua Water Technologies). Meso-tetra (4-pyridyl) porphine was purchased from Frontier Sciences and used without further purification. NMR were recorded on Bruker 300 MHz NMR, and FTIR on a Bruker alpha ATR-FTIR with the bare ATR crystal in open air as the background.

### Example 2

Synthesis of the manganese (III) meso-tetrakis-(1-methylpyridinium-4-yl) porphyrin pentaacetic acid (MnP): The carboxyl monofunctionalized tetrapyridyl manganese (III) porphyrin was synthesized according to a modified procedure (Jacobs et al., (1997) J. Org. Chem. 62: 3505-3510, incorporated herein by reference in its entirety). Briefly, 1 g meso-tetra (4-pyridyl) porphine (1) was refluxed with 4.78 g ethyl bromopentanoate (approximately 14 eq) in 300 mL of 25% (v/v) ethanol/CHCl₃ for 7 days. The solvent was evaporated and the resulting 5-(1-(4-(ethoxycarbonyl)butyl)pyridinium-4-yl)10,15,20-tripyridylporphyrin bromide (2) was dried under vacuum at 50° C. for 24 h. Then, 1.06 g of unpurified 2 was added to 50 mL DMSO and brought to 42° C. with 1.25 mL methyl iodide (CH₃I). After 2 h, another 1.25 mL CH₃I was added. After another 3 h, the solvent was evaporated under vacuum and the alkylated product, tetracationic porphyrin (3), was dried in a vacuum oven at 50° C. for 24 h. To hydrolyze the ester function, 750 mg of 3 was added to 70 mL of 1 M HCl and refluxed for 3 h. The mixture was cooled to room temperature and filtered through a sintered glass funnel. The filtrate was evaporated and washed with DI water three times. For metalation of the obtained meso-tetrakis-(1-methylpyridinium-4-yl) porphyrin monopentaacetic acid (4) with manganese (II) chloride, 54 mg of 4 was added to 5 mL deionized water along with 30 mg manganese (II) chloride tetrahydrate and refluxed for 80 min. The metalation to the corresponding manganese (III) meso-tetrakis-(1-methylpyridinium-4-yl) porphyrin pentaacetic acid (5) was confirmed by UV-visible spectroscopy (Varian Cary Bio50). The mixture was cooled to room temperature, precipitated in 50% (v/v) ethyl ether/isopropanol and centrifuged to collect the precipitated carboxyl monofunctionalized tetrapyridyl manganese (III) porphyrin (MnP).

### Example 3

Synthesis of MnP-functionalized poly(N-vinylpyrrolidone) (MnP-PVPON): To obtain MnP-PVPON, the PVPON-NH₂-20% copolymer (20% represents the molar percentage of amino-group-containing polymer units) was synthesized using gradual feeding copolymerization of VPON and tBOC as we reported previously (Rahim et al., (2016) Angew. Chem. Int. Ed. 55: 13803-13807). For example, 6.3 g of freshly distilled VPON in dioxane (25 mL) was degassed with three freeze-pump-thaw cycles. N-(t-BOC-aminopropyl)methacrylamide (tBOC, 2.404 g) was dissolved in dioxane (12 mL), filtered through a 0.2-μm syringe filter, and diluted to 25 mL. AIBN (30 mg) in 1 mL dioxane was added to the VPON solution, and the solution was heated to 70° C. under nitrogen and constant stirring for 3.5 h during which the tBOC solution was gradually added using a syringe pump. After that, the mixture was cooled to room temperature and the PVPON-tBOC copolymer was precipitated in petroleum ether, collected and purified by dissolving and precipitating in dichloromethane and petroleum ether, respectively, two times. The tBOC groups were hydrolyzed from PVPON-tBOCc (210 mg) in methanol (17 mL) containing hydrochloric acid (3 mL) for 3 days, followed by neutralizing the acid with 1 M NaOH. The PVPON-NH₂ copolymer was dialyzed against deionized water using a Spectra/Por Float-A-Lyzer with a MWCO of 50,000 Da and freeze-dried (Labconco). The composition of the PVPON-NH₂ random copolymer after the hydrolysis was determined with ¹H NMR in D₂O. The molecular weight of the copolymer (Mw=198,000 g mol⁻¹, Ð=1.2) was determined with GPC (Waters) in dimethylformamide using linear polystyrene standards. The PVPON-NH₂-20 copolymer (135 mg) dissolved in 10 mL 0.01 M phosphate buffer (pH=6) was added to an MnP solution which was prepared by dissolving 123 mg MnP and 41 mg EDC in 6 mL 0.01 M phosphate buffer (pH=6) and stirring for 30 min. The polymer solution was stirred overnight in the dark, followed by exhaustive dialysis against 0.01 M phosphate buffer (pH=8, 0.1 M NaCl), and DI water (MWCO 20,000 Da) to remove free porphyrin until no trace of porphyrin color was observed in the external solution. The MnP polymer solution was freeze-dried and stored in the dark.

### Example 4

Synthesis of (TA/PVPON) and (TA/PVPON/TA/MnP-PVPON) and (PVPON)₆ multilayer capsules: Hollow hydrogen-bonded multilayer spherical capsules were prepared by coating 4 μm solid silica particles with (PVPON/TA)ₙ multilayers via alternating exposure of the particles to 0.5 mg mL⁻¹ PVPON, TA and MnP-PVPON polymer solutions (0.01 M phosphate buffer, pH=5) followed by particle dissolution, where “n” denotes the number of (PVPON/TA) bilayers. Briefly, 40 mg of silica cores suspended in 1 mL of phosphate buffer (0.01 M, pH=5) were pelleted in a 1.5 mL Eppendorf centrifuge tube and re-suspended in PVPON solution. The polymer was allowed to deposit on the surface for five minutes followed by rinsing with the phosphate buffer (0.01 M, pH=5) followed by adsorption of TA for 5 min. Three rinses were applied after each layer's deposition. Alternating multilayer deposition was performed until the desired number of polymer layers was achieved. The following multilayer architectures were created: (PVPON/TA)₅; (PVPON/TA)_{5.5}; and (MnP-PVPON/TA)₂(PVPON/TA)_{3.5}, (PVPON/TA)₂(MnP-PVPON/TA)₂(PVPON/TA)_{1.5}, and (PVPON/TA)₄(MnP-PVPON/TA)_{1.5}. The porphyrin-containing capsules, referred to as S1, S2, and S3, respectively, the number denoting the location of the MnP-containing bilayer in the inner, middle, or outer part of the (PVPON/TA) multilayer shell were covered or kept in the dark. Silica cores were dissolved in hydrofluoric acid (8% wt) to yield hollow polymeric capsules. The capsules were dialyzed against 0.01 M phosphate buffer (0.1 M NaCl, pH=8) for two days, followed by DI water dialysis for two days using 1 mL Float-A-Lyzers (MWCO 20,000 Da, Spectrum Laboratories). The TA-free (PVPON)₆ capsules were prepared as previously described (Kozlovskaya et al., (2015) Adv. Healthcare Mater. 4: 686-694). Hydrogen-bonded multilayers of poly(methacrylic acid) (PMAA) with amino-containing poly(N-vinylpyrrolidone) (PVPON-NH₂-7, Mw=143884 g mol⁻¹, Ð=1.55) copolymer with a 7% molar percentage of amino group-containing polymer units were deposited on the silica particles. Assembly of the hydrogen-bonded layers was performed at pH=3, starting from PVPON-NH₂ followed by PMAA. Each deposition cycle was followed by rinsing three times with a buffer solution at pH=3 to remove excess polymer, followed by centrifugation of suspensions at 2000 rpm for 2 min to remove supernatant. After six bilayers of (PVPON-NH₂/PMAA) were deposited, chemical cross-linking of PVPON-NH₂ was performed. For that, the core-shell particles were exposed to glutaric aldehyde solution (25 wt %) at pH=5 for 12 hours. After that, core dissolution was performed as described above resultant (PVPON)₆ capsules were purified by dialysis in de-ionized water for 3 days. For accurate capsule concentrations in all experiments, capsule suspensions were counted (capsules mL⁻¹) using a hemocytometer and an optical microscope with an 40× objective.

### Example 5

Nuclear magnetic resonance (NMR): ¹H NMR spectra were recorded on a Bruker 300 MHz NMR spectrometer. The porphyrin solutions (1 mg mL⁻¹) in (CD₃)₂SO (Sigma-Aldrich), and PVPON-NH₂ copolymer in D₂O were measured at 25° C.

### Example 6

Scanning electron microscopy (SEM): SEM was performed using a FEI Quanta® FEG microscope at 10 keV. Samples were prepared by depositing a drop of a capsule suspension on a silicon wafer and allowing it to dry at room temperature. Before imaging, dried specimens were sputter-coated with 5 nm silver film using a Denton sputter-coater.

### Example 7

Wettability measurements: Thin film wettability was assessed by contact angle measurements with a Theta Lite 101 tensiometer (Biolin Scientific) using a sessile drop experiment. A 5 mL drop of deionized water was placed on the film surface at room temperature and the contact angle was acquired with a CCD camera and analyzed using Attension software. The measurements were done in five different spots and the average contact angle value and its standard deviation were calculated.

### Example 8

Ellipsometry: Film thickness was measured using a M2000U spectroscopic ellipsometer (Woollam). Single-side polished silicon wafers 500-μm-thick (University Wafer) of 20 mm×50 mm were cleaned in a mixture of concentrated sulfuric acid and Nocromix (Godax Laboratories) for 24 hours, washed with DI water, dried with a stream of nitrogen (Airgas) and used immediately thereafter. First, a precursor layer of poly(ethyleneimine) (PEI, Sigma-Aldrich) was adsorbed onto the wafer surface from 1 mg mL⁻¹ PEI aqueous solution for 10 min. After that the deposition of TA, PVPON and MnP-PVPON onto silicon wafers coated with a PEI layer was performed using a 5-mL liquid flow-through cell (Woollam) at pH=5 and pH=7.2 (0.01 M phosphate buffer). The pH values of 0.01 M phosphate buffers were adjusted using 0.1 M HCl and NaOH aqueous solutions. Dry thickness measurements were performed between 400 and 1000 nm at 65, 70, and 75° angles of incidence, while wet thickness studies were monitored at 75° incidence angle. For data interpretation, the ellipsometric angles Ψ and Δ were fitted using a multilayer model composed of silicon, silicon oxide, and the multilayer film to obtain the thickness of films. The thickness of silicon oxide was determined using known optical constants. The thickness of the dry multilayer film was obtained by fitting data with the Cauchy approximation with the refractive index as

$$n ( λ ) = {A}_{n} + \frac{{B}_{n}}{{λ}^{2}} + \frac{{C}_{n}}{{λ}^{4}} ,$$

with Aₙ=1.5, Bₙ=0.01, and Cₙ=1.3×10⁻⁵. The thickness of the in-situ grown film was obtained by fitting data with the Cauchy approximation with permitted fitting of Aₙ, Bₙ, and Cₙ. The mean squared error for data fitting was less than 30.

### Example 9

Free radical scavenging activity of (TA/PVPON) and (TA/MnP-PVPON) capsules using 2,2-azinobis(3-ethylbenzothiazoline-6-sulfonic acid) radical cation (ABTS⁺⁻) assay: ABTS⁺⁻ was prepared according to a previously published procedure (Shutova et al., (2007) Russ. J. Gen. Chem. 77: 1494-1501). ABTS was dissolved in DI water at a concentration of 0.14 M and treated with 100 μL of a 0.12 M solution of potassium persulfate in DI water. The mixture was left in the dark at room temperature overnight before being stored at 3° C. Before use, the solution was diluted to reach an absorbance of 1.2 at 734 nm. 100 μL of a 1×10⁸ capsule mL⁻¹ suspension (0.01 M phosphate buffer, pH=7.4) was added to a UV-vis quartz cuvette containing the dye, and mixed while recording the absorbance at 734 nm every 30 s for 20 min.

### Example 10

Superoxide dismutation by cytochrome C assay: A steady stream of superoxide radicals was produced in situ over 15 min through a xanthine/xanthine oxidase reaction and monitored via the reduction of cytochrome C (detected at 550 nm) (Arai et al., (2012) ACS Appl. Mater. Interfaces 4: 5453-5457). The rate of superoxide dismutation was followed in 0.05 M phosphate buffer with 0.1 mM EDTA at pH=7.8 in a 1-cm UV-vis quartz cuvette in the presence of (PVPON/TA)_{5.5} or (PVPON/TA)₄(MnP-PVPON)_{1.5} capsules and in the presence and absence of 10 μg/mL catalase. Absorbance at 550 nm from 10 μM cytochrome C was used as the radical indicator in the presence or absence of 10 μg mL⁻¹ catalase. The xanthine stock was oxygenated before each use and the experiments were carried out at 23° C. 100 μL of a 1×10⁸ capsule mL⁻¹ suspension (0.01 M phosphate buffer, pH=7.4) was added to a UV-vis quartz cuvette containing the radical, and mixed while recording the absorbance at 550 nm every 15 s for 15 min.

### Example 11

Small angle neutron scattering (SANS): SANS measurements were carried out at the CG3 Bio-SANS beamline at the High Flux Isotope Reactor (HFIR) in Oak Ridge National Laboratory. A constant neutron wavelength of 6 Å with a relative wavelength spread of 15% was used in all measurements. One single instrument configuration was able to cover the q range of 0.003 to 0.7 Å⁻¹ by utilizing both a main and wing detector. The scattering intensity profiles I(q) versus q were obtained by azimuthally averaging the processed 2D images, which were normalized using water as a secondary standard, and corrected for detector dark current, pixel sensitivity and scattering from backgrounds (D₂O and quartz cell). The capsules were suspended in D₂O before being placed in titanium tumbler cells with quartz windows. The tumbler cells were rotated constantly during the scans to prevent the capsules from settling. ABTS was activated with potassium persulfate in D₂O overnight in accordance with the procedure used in the UV-vis experiments. The (PVPON/TA)_{5.5} and (PVPON/TA)₄(MnP-PVPON-TA)_{1.5} capsules were scanned in the Bio-SANS instrument before being added to the ABTS⁺⁻ solution and again after the reaction had gone to completion to measure shell thickness change. For data analysis, scattering curves were fitted using the LamellarFF model (Berghausen et al., (2001) J. Phys. Chem. B 105: 11081-11088), where the scattering intensity is returned; scale*I(q)+incoherent bkg and I(q) is given by

$$I ( q ) = \frac{4 {πΔρ}^{2}}{δ {q}^{4}} [ 1 - cos ( q δ ) {e}^{- {q}^{2} {σ}^{2} \text{/} 2} ]$$

where q=(4π/λ)sin(θ/2), λ is the neutron wavelength, θ is the scattering angle, δ is shell thickness, σ is variation in shell thickness, and Δρ² is the contrast factor (the difference between the scattering length densities (SLD) of the solvent and shell material). The data were analyzed with Igor Pro using a macro provided by National Institute of Standards and Technology (Kline, S. R. (2006) J Appl. Cryst. 39: 895). The fitting parameters are presented in Tables S1-S4 of the Supporting Information. The Lamellar model (instead of the core-shell model) was applied due to the following considerations: using the equation for R_{g} of a solid sphere R_{g}²=3R²/5, the minimum q needed to resolve size information on 4 μm spheres would be q˜0.0001 (Guinier scattering at qRg<1.3), which is beyond the lower limit of the instrument. However, at higher q, Porod surface scattering of a locally flat sheet can be modeled with a lamellar bilayer. Since the capsule wall effectively scatters as a flat sheet at the q range covered in our experiment, the lamellar model can be used to extract the shell thickness and ignore the size parameter which is easily resolved by standard optical microscopy. The starting values of the shell thicknesses were taken from ellipsometry measurements and adjusted to fit the scattering curves. Fit quality was determined by minimizing goodness-of-fit parameter (χ²), and parameters were constrained to physically reasonable values of thickness and consistency with other measurements, such as ellipsometry and atomic force microscopy.

### Example 12

Mice: NOD/ShiLtJ mice were bred and housed under pathogen free conditions. Female mice between 8 and 16 weeks of age were used. Mice received standard chow.

### Example 13

Cell viability: An MTT cell viability assay was performed according to the manufacturer's protocol (Sigma Aldrich). Splenocytes from NOD mice were purified and cultured in 96-well plates at 5.0×10⁶ cells μL⁻¹. Each plate contained quadruplicates of control and capsule-treated (2.5×10⁶ or 5.0×10⁶ counts μL⁻¹) splenocytes incubated for 48 hours. Absorbance readings were analyzed on a Synergy 2 microplate reader (BioTek) using Gen5 software. Percentage cell viability was determined by subtracting the absorbance reading from the blank with all samples and then dividing the test sample with the control and multiplying by 100.

### Example 14

Statistical analysis: Data were analyzed using GraphPad Prism Version 5.0 statistical software. Determination of the difference between mean values for each experimental group was assessed using the 2-tailed Student's t test, with p<0.05 considered significant. All experiments were performed at least three separate times with data obtained in quadruplicate wells for each experiment.

### Example 15

TABLE 2
SANS model parameters for (PVPON/TA)_{5.5}
capsules before ABTS treatment.

| Parameter | Value | Wσ |
|---|---|---|
| Scale | 0.04189888 | 0 |
| Shell Thickness (δ) (Å) | 181.01 | 1.516424227 |
| Polydispersity of Thickness (0,1) | 0.90920791 | 0.009720238 |
| SLD shell (Å⁻²) | 3.97E−06 | 0.003735086 |
| SLD solvent (Å⁻²) | 4.13E−06 | 0.003735089 |
| Background (cm⁻¹) | 0.009165115 | 4.47E−05 |

| | |
|---|---|
| Reduced X² | 1.56 |

### Example 16

TABLE 3
SANS model parameters for (PVPON/TA)_{5.5}
capsules after ABTS treatment.

| Parameter | Value | Wσ |
|---|---|---|
| Scale | 0.000824393 | 0 |
| Shell Thickness (δ) (Å) | 110.011 | 0.70027147 |
| Polydispersity of Thickness (0,1) | 0.98 | 0 |
| SLD shell (Å⁻²) | 4.81E−06 | 3.11E−10 |
| SLD solvent (Å⁻²) | 6.03E−06 | 3.11E−10 |
| Background (cm⁻¹) | 0.005 | 0.000105174 |

| | |
|---|---|
| Reduced X² | 729 |

### Example 17

TABLE 4
SANS model parameters for (PVPON/TA)₄(MnP-
PVPON/TA)_{1.5} capsules before ABTS treatment.

| Parameter | Value | Wσ |
|---|---|---|
| Scale | 0.000199736 | 0 |
| Shell Thickness (δ) (Å) | 152.78 | 1.440568096 |
| Polydispersity of Thickness (0,1) | 0.671286802 | 0.012447488 |
| SLD shell (Å⁻²) | 1.89E−06 | 0.003867379 |
| SLD solvent (Å⁻²) | 3.80E−06 | 0.005586294 |
| Background (cm⁻¹) | 0.010648118 | 3.56E−05 |

| | |
|---|---|
| Reduced X² | 1.60 |

### Example 18

TABLE 5
SANS model parameters for (PVPON/TA)₄(MnP-
PVPON/TA)_{1.5} capsules after ABTS treatment.

| Parameter | Value | Wσ |
|---|---|---|
| Scale | 0.000228364 | 0 |
| Shell Thickness (δ) (Å) | 140.84 | 1.097048584 |
| Polydispersity of Thickness (0,1) | 0.64706398 | 0.009723314 |
| SLD shell (Å⁻²) | 1.77E−06 | 0.002732288 |
| SLD solvent (Å⁻²) | 3.92E−06 | 0.003898266 |
| Background (cm⁻¹) | 0.028858531 | 7.43E−05 |

| | |
|---|---|
| Reduced X² | 1.44 |

What is claimed:

1. A multilayered composition comprising a plurality of polymer bilayers, wherein each polymer bilayer comprises a poly(N-vinylpyrrolidone) (PVPON) polymer layer and a polyphenolic tannin layer, wherein the poly(N-vinylpyrrolidone) (PVPON) polymer layer and the polyphenolic tannin layer are hydrogen-bonded, and wherein at least one poly(N-vinylpyrrolidone) (PVPON) layer comprises a plurality of manganoporphyrin moieties conjugated to the poly(N-vinylpyrrolidone) (PVPON) layer.

2. The multilayered composition of claim 1, wherein the multilayered composition is configured as a multi-layered capsule defining a volume or is disposed as a multilayered coating on a support surface.

3. The multilayered composition of claim 1, comprising between about 3 polymer layers and about 10 polymer layers.

4. The multilayer composition of claim 2, wherein a polymer layer proximal to the defined volume or proximal to the support surface is a poly(N-vinylpyrrolidone) (PVPON) layer.

5. The multilayered composition of claim 1, wherein at least one poly(N-vinylpyrrolidone) (PVPON) layer comprising a plurality of manganoporphyrin moieties conjugated thereto is embedded within the multilayered composition.

6. The multilayered composition of claim 2, wherein the composition further comprises a poly(N-vinylpyrrolidone) (PVPON) layer disposed on the plurality of polymer bilayers as a layer most distal from the defined encapsulated volume or from the support surface and further comprising a plurality of manganoporphyrin moieties conjugated thereto.

7. The multilayered composition of claim 2, further comprising an animal cell or aggregate of animal cells within the volume defined by the multi-layered capsule.

8. The multilayered composition of claim 7, wherein the polymer layer of the multi-layered capsule proximal to the defined volume of the capsule is hydrogen bonded to the cell membrane of a cell or a plurality of cells within the volume defined by the multi-layered capsule.

9. The multilayered composition of claim 1, wherein at least one polymer layer of the multilayered composition further comprises a functional moiety attached thereto, and wherein the functional moiety is selected from the group consisting of: a detectable moiety, an immunomodulatory molecule, a growth factor, a cell receptor ligand, a polypeptide cell receptor, or any combination thereof, and wherein the at least one polymer layer optionally comprises a molecular group suitable for attaching the functional moiety to the polymer layer.

10. The multilayered composition of claim 2, wherein the multi-layered capsule further comprises a pharmacologically active compound that reacts with a reactive oxygen species when delivered to a recipient animal or human subject.

11. The multilayered composition of claim 10, wherein the pharmacologically active compound is a therapeutic agent.

12. The multilayered composition of claim 1 admixed with a pharmaceutically acceptable carrier.

13. A method of encapsulating an isolated cell or aggregate of cells, the method comprising the steps of:
(a) providing an isolated cell or cell aggregate; and
(b) encapsulating the isolated cell or cell aggregate by depositing a plurality of polymer bilayers, wherein each polymer bilayer comprises a poly(N-vinylpyrrolidone) (PVPON) polymer layer and a polyphenolic tannin layer, wherein the poly(N-vinylpyrrolidone) (PVPON) polymer layer and the polyphenolic tannin layer are hydrogen-bonded, and wherein at least one poly(N-vinylpyrrolidone) (PVPON) layer comprises a plurality of manganoporphyrin moieties conjugated to the poly(N-vinylpyrrolidone) (PVPON) layer, wherein the deposited plurality of polymer bilayers forms a capsule encapsulating the isolated cell or cell aggregate.

14. The method of claim 13, wherein the polymer layer in contact with the isolated cell or cell aggregate is poly(N-vinylpyrrolidone) hydrogen-bonded to the outer surface of the cell or aggregate of cells, or to an extracellular matrix component of the cell aggregate.

15. The method of 13, wherein the method further comprises the step of attaching at least one functional moiety to the capsule encapsulating the cells or cell aggregate.

16. The method of 15, wherein the functional moiety is selected from the group consisting of: a detectable moiety, an immunomodulatory molecule, a growth factor, a cell receptor ligand, a polypeptide cell receptor, or any combination thereof.

17. The method of 13, wherein the cell or cell aggregate is selected from the group consisting of: a bacterial cell, a viral cell, a plant cell, an artificial cell, and an animal cell, and wherein the cell or aggregate of cells is an isolated cell or aggregate of cells or a cultured cell or aggregate of cells.

18. The method of claim 17, wherein the cell aggregate is an isolated human or animal pancreatic islet or population of islets.

19. The method of claim 18, wherein the pancreatic islet or population of islets is dissected from a pancreas or comprises cultured pancreatic islet cells.

20. An encapsulated cell aggregate, wherein the cell aggregate is coated with a plurality of polymer bilayers, wherein each polymer bilayer comprises a poly(N-vinylpyrrolidone) (PVPON) polymer layer and a polyphenolic tannin layer, wherein the poly(N-vinylpyrrolidone) (PVPON) polymer layer and the polyphenolic tannin layer are hydrogen-bonded, and wherein at least one poly(N-vinylpyrrolidone) (PVPON) layer comprises a plurality of manganoporphyrin moieties conjugated to the poly(N-vinylpyrrolidone) (PVPON) layer, wherein the deposited plurality of polymer bilayers forms a capsule encapsulating the isolated cell or cell aggregate.

21. The coated aggregate of cells of claim 20, wherein the cell aggregate is an isolated pancreatic islet dissected from a pancreas or is a cultured pancreatic islet, and wherein the coated aggregate of cells is capable of producing insulin when the coated aggregate is transplanted into a recipient human or animal subject.

22. The coated aggregate of cells of claim 20, wherein the capsule further comprises a functional moiety attached thereto.

23. The coated aggregate of cells of claim 22, wherein the functional moiety is selected from the group consisting of: a detectable moiety, an immunomodulatory molecule, a growth factor, a cell receptor ligand, a polypeptide cell receptor, or any combination thereof.
